SEQUENCE LISTING (18 sequences)

SEQ ID NO: 1 - protein, 322 aa, Erinaceus europaeus
GKIAREPLTNYLDTEYFGKIQIGSPPQEFTVVFDTGSSDLWVPSVFCNSDACQNHHRFNPSKSSTFQNMNRPLSIQYGTGSMQGFLGYDTVTVSNIVVPHQTVGLSTQEPGDIFTYSEFDGILGLAYPSLASQYSVPVFDNMMKQHLVAQDLFSVYMNSQGQGSMLILGAIDPSYYVGALHWVPVTVQQYWQFTVDRITVDGVVVACDGGCQAILDTGTSLLVGPSSDILNIQTVIGATQGQYGEFDIDCGSLRSKPTVVFEINGREYPLPPTAYTNQDQGFCTSGFQGDDSQQWVLGDVFIREYYSVFDRVNNRVGLAKAI

SEQ ID NO: 2 - protein, 323 aa, Saimiri boliviensis
GKVARESLTNYLDCQYFGKIYIGTPPQEFTVVFDTGSSDLWVPSVYCNSDACQNHHRFDPSKSSTFQNMDKSLSIQYGTGSMQGLLGYDTVTVSNIVDRHQTVGLSTQEPGDVFTYSEFDGILGLAYPSLASEYSVPVFDNMMDRHLVAQDLFSVYMSRNEQGSMLTLGAIDRSYYTGSLHWIPVTVQEYWQFTVDRVTVDGVVVACDGGCQAILDTGTSMLVGPGSDIFNIQQAIGATEGQFGEFDIDCGRLSSMPTVVFEINGKKYPLPPSAYTSQDQGFCTSGFQGDYSSQQWILGDVFIREYYSVFDRASNRVGLAKAI

SEQ ID NO: 3 - protein, 326 aa, Monodelphis domestica
GEAAKVTDEPLTNYLDSQYFGKIYIGTPPQEFTVVFDTGSSNLWVPSVYCNSDACQNHHRFNPASSTTFRSTQEPLSIQYGTGSMEGVLGYDTVTVSQIVVPDQIFGLSTQEPGEIFTYSEFDGILGLGYPSLAEDQATPVFDNMMNKNLVAQDLFSVYMSRDSQGSMLILGAIDPSYYTGSLHWVPVTEQGYWQFSVDSITVNGQVVACEGGCQAILDTGTSLLVGPSYDIANIQSIIGATQGQYGEYDINCSNLSSMPTVVVHINGRQYPLPPSAYTNQDQGLCSSGFQSEGSDQLWILGDVFIREYYSVFDRGNNRVGLATAV

SEQ ID NO: 4 - protein, 323 aa, Orcinus orca
GEVASEPLTSYLDSQYFGKIYIGTPPQEFTVVFDTGSSDLWVPSVYCKSDACQNHQRFDPSMSSTFQNLGKPLSIQYGTGSMQGFLGYDTVTVSDIVDPQQTVGLSTQEPGDVFTYSEFDGILGLAYPSLASEYSVPVFDNMMNRHLVAQDLFSVYMDRNGQESMLTLGAIDPSYYTGSLHWVPVTLQKYWQFTVDSVTIGGVVVACDGGCQAILDTGTSMLVGPSSDILNIQMAIGATQNQYGEFDIDCGSLSSMPTVVFEINSRMYPLTPSAYTNQDQGFCTSGFQGENNSQQWILGDVFIREYYSVFDRANNRVGLAKAV

SEQ ID NO: 5 - protein, 361 aa, Erinaceus europaeus
SGITRVPLLKGKSLRKSLKEHGLLEEFLQTHARKKLAHSGKIAREPLTNYLDTEYFGKIQIGSPPQEFTVVFDTGSSDLWVPSVFCNSDACQNHHRFNPSKSSTFQNMNRPLSIQYGTGSMQGFLGYDTVTVSNIVVPHQTVGLSTQEPGDIFTYSEFDGILGLAYPSLASQYSVPVFDNMMKQHLVAQDLFSVYMNSQGQGSMLILGAIDPSYYVGALHWVPVTVQQYWQFTVDRITVDGVVVACDGGCQAILDTGTSLLVGPSSDILNIQTVIGATQGQYGEFDIDCGSLRSKPTVVFEINGREYPLPPTAYTNQDQGFCTSGFQGDDSQQWVLGDVFIREYYSVFDRVNNRVGLAKAI

SEQ ID NO: 6 - protein, 365 aa, Saimiri boliviensis
SIISRIPLHKGKSLRKALKERGLLEDFLKNHQYAVSRKHSSSGKVARESLTNYLDCQYFGKIYIGTPPQEFTVVFDTGSSDLWVPSVYCNSDACQNHHRFDPSKSSTFQNMDKSLSIQYGTGSMQGLLGYDTVTVSNIVDRHQTVGLSTQEPGDVFTYSEFDGILGLAYPSLASEYSVPVFDNMMDRHLVAQDLFSVYMSRNEQGSMLTLGAIDRSYYTGSLHWIPVTVQEYWQFTVDRVTVDGVVVACDGGCQAILDTGTSMLVGPGSDIFNIQQAIGATEGQFGEFDIDCGRLSSMPTVVFEINGKKYPLPPSAYTSQDQGFCTSGFQGDYSSQQWILGDVFIREYYSVFDRASNRVGLAKAI

SEQ ID NO: 7 - protein, 367 aa, Monodelphis domestica
AFRRIPLTKGKTLRKVLKEHGLLESFLKSHKYSPSSKYQLYGEAAKVTDEPLTNYLDSQYFGKIYIGTPPQEFTVVFDTGSSNLWVPSVYCNSDACQNHHRFNPASSTTFRSTQEPLSIQYGTGSMEGVLGYDTVTVSQIVVPDQIFGLSTQEPGEIFTYSEFDGILGLGYPSLAEDQATPVFDNMMNKNLVAQDLFSVYMSRDSQGSMLILGAIDPSYYTGSLHWVPVTEQGYWQFSVDSITVNGQVVACEGGCQAILDTGTSLLVGPSYDIANIQSIIGATQGQYGEYDINCSNLSSMPTVVVHINGRQYPLPPSAYTNQDQGLCSSGFQSEGSDQLWILGDVFIREYYSVFDRGNNRVGLATAV

SEQ ID NO: 8 - protein, 365 aa, Orcinus orca
TGITRIPLHKGKPLRKALKEHGLLEDFLQKHQSAVSSKYSSFGEVASEPLTSYLDSQYFGKIYIGTPPQEFTVVFDTGSSDLWVPSVYCKSDACQNHQRFDPSMSSTFQNLGKPLSIQYGTGSMQGFLGYDTVTVSDIVDPQQTVGLSTQEPGDVFTYSEFDGILGLAYPSLASEYSVPVFDNMMNRHLVAQDLFSVYMDRNGQESMLTLGAIDPSYYTGSLHWVPVTLQKYWQFTVDSVTIGGVVVACDGGCQAILDTGTSMLVGPSSDILNIQMAIGATQNQYGEFDIDCGSLSSMPTVVFEINSRMYPLTPSAYTNQDQGFCTSGFQGENNSQQWILGDVFIREYYSVFDRANNRVGLAKAV

SEQ ID NO: 9 - DNA, 966 bp, Synthetic construct
  source 1..966: mol_type = other DNA
gggaagatcgcgcgagagccgctcacaaattatttggacacggagtacttcggcaaaatccagatcggtagcccgcctcaagagtttacagtcgtgttcgacaccggttcttcggatctctgggttccttccgtcttttgcaatagtgatgcctgtcagaaccatcatcgctttaatccatccaagtctagcaccttccaaaacatgaaccgccccctgagtatccaatacggcaccggtagtatgcaggggttcttgggttatgatacagtcaccgtatcgaacatcgtggttccacaccaaaccgtaggattgagcactcaggaacctggcgacattttcacttactccgaattcgatggcatcctcggactggcgtacccgtccctggcttcacaatattctgtgcccgtctttgacaacatgatgaagcagcacctcgtggctcaagaccttttctctgtttatatgaatagccagggacagggttccatgctgatactgggggctattgacccctcatactacgtcggagcacttcattgggtccctgtgacggttcagcagtattggcagttcaccgtcgaccggataaccgtggatggagttgtcgtggcctgcgacggaggttgtcaagcgattcttgataccggcacatcactcctcgttgggccctcctctgatatattgaacatccagacggttatcggcgcaacccagggccaatacggggaattcgacattgattgcggttccctgagatcgaagccaacggtggtattcgagattaatggtcgcgaatacccgctcccgcccactgcttatactaatcaggaccagggcttctgcacttcgggttttcaaggagatgatagccagcaatgggttcttggcgacgtgtttatccgggaatactattccgtatttgatcgtgtcaacaacagggtcggcctggcaaaggccatt

SEQ ID NO: 10 - DNA, 969 bp, Synthetic construct
  source 1..969: mol_type = other DNA
gggaaggtcgcaagagagagccttaccaactacctggattgccagtacttcgggaagatatacattggcacaccgccccaagagttcaccgtggttttcgacactggcagttcagacctctgggtgccatctgtctactgcaattctgatgcctgtcagaatcatcatcgcttcgacccgtctaagtcgtccacatttcaaaacatggataagtcgctgtctatccaatatggtaccggttcgatgcagggtcttttgggctatgacacagttactgtctcaaatattgtcgatcgccaccagacggtcggcctgagcacccaagagcccggcgacgtgttcacttactctgaattcgatggcatcctcggactggcctacccctctctcgcgagcgaatactcggtaccagtgttcgataacatgatggataggcacctggtcgcccaagatctcttctccgtctatatgagtcggaacgagcagggttccatgttgactctcggggctatagatcgatcctattacacggggtcgttgcattggatcccagtgacggttcaggagtactggcagtttacggtcgatcgggttaccgtggacggcgtggtcgtagcctgtgatggcggatgccaggctattttggacaccggtaccagcatgctcgtgggaccgggttcagacattttcaatattcagcaagccatcggtgcaaccgagggacaatttggggagttcgacatcgattgcggcagacttagctctatgcccactgttgtctttgaaatcaacgggaaaaagtaccctctccctccttccgcttatacgagtcaggaccagggcttttgtacatcgggttttcaaggagattattcaagccagcaatggattcttggcgacgtattcatacgtgaatactattccgttttcgaccgcgcaagcaaccgtgttggattggcgaaagctatc

SEQ ID NO: 11 - DNA, 978 bp, Synthetic construct
  source 1..978: mol_type = other DNA
ggggaagcagctaaggtcaccgacgaacccctgacaaattacctcgactcgcaatactttggcaaaatctacattgggacaccaccgcaggaattcactgtcgtatttgataccggctcatccaacctttgggtgccgtctgtctactgcaattcggatgcgtgtcagaaccatcaccgattcaatccagcctcgtctactaccttccgatcaacccaggagccgctcagtattcagtatggcaccggctcaatggaaggcgtcctcggttacgatacagtcactgtcagtcaaattgttgtgcctgatcagatcttcggactctcgacccaagagcctggggagatcttcacgtactccgagttcgacgggatcctgggtctgggatacccctccttggcggaggatcaggccactccagttttcgacaatatgatgaacaaaaacctcgtggcccaagatctcttctctgtctatatgagcagggactcccagggttcgatgctgattcttggcgcgatagatccctcctattacacgggttcactgcattgggtgcccgttacggagcagggatattggcagttcagtgtcgattcaattaccgtcaacggccaggttgtggcttgtgaaggcggttgtcaagctatcttggacactggcacaagcctcctcgttggaccgagctacgatatcgccaacatccaatccattatcggagccacccagggccaatacggagaatatgacatcaactgcagcaacctgagcagtatgcccacggtggtagtccacataaacgggcgtcaatacccacttcctccctccgcgtatactaatcaggatcagggtttgtgctcctccggttttcaatctgagggctcagatcagttgtggatcttgggggacgtgtttataagagagtactactcggtgttcgaccggggaaacaatcgcgtcggtctggcaacggcagta

SEQ ID NO: 12 - DNA, 969 bp, Synthetic construct
  source 1..969: mol_type = other DNA
ggggaagtagcttcagagccgttgacgtcatacctggacagtcagtacttcggaaagatctacatcggaacgccgccccaggaattcaccgtcgttttcgacactggctcctcggatttgtgggtaccctctgtttactgcaaaagcgatgcatgccaaaatcatcagcgttttgatccatcaatgtcgagtacattccagaatctcgggaagcccttgtctatccaatacggcaccggatctatgcagggcttcctcggttatgataccgtcactgtgtccgacatagtcgatccacaacagactgtcggcctctccacacaagagcctggggatgtgttcacttactcggaatttgacggaatcttgggcctggcgtatccgagcctcgcttccgaatactccgtccccgttttcgataacatgatgaatcggcatctggttgctcaagacctcttctccgtctatatggatcgaaatggacaagagtcgatgcttacgctgggagctatcgacccgagctactatactggatctctccactgggtccctgtgacgcttcaaaagtactggcagtttaccgtcgacagtgtgacgatcggtggtgttgtcgttgcctgcgacggcggttgccaagcgattctggataccggcacctcaatgcttgtgggcccttcgagtgatattctgaacatccagatggcgataggggccacacagaaccaatatggggagttcgacattgactgtggttcgcttagttcgatgcccaccgtggtgttcgagatcaactcacgcatgtacccgctcacacctagcgcatatacaaatcaggaccagggtttctgtacaagcggttttcagggagaaaacaattcccagcaatggatccttggggacgtattcataagggagtattactctgtgtttgatcgggccaacaacagagtcggtctggctaaggccgtt

SEQ ID NO: 13 - DNA, 1083 bp, Synthetic construct
  source 1..1083: mol_type = other DNA
tcgggcatcacacgagtgccacttctgaaagggaagtcgttgagaaagtcactcaaagagcacggactcttggaggagttcctgcaaacgcatgcccgtaaaaagctcgcccactctgggaagatcgcgcgagagccgctcacaaattatttggacacggagtacttcggcaaaatccagatcggtagcccgcctcaagagtttacagtcgtgttcgacaccggttcttcggatctctgggttccttccgtcttttgcaatagtgatgcctgtcagaaccatcatcgctttaatccatccaagtctagcaccttccaaaacatgaaccgccccctgagtatccaatacggcaccggtagtatgcaggggttcttgggttatgatacagtcaccgtatcgaacatcgtggttccacaccaaaccgtaggattgagcactcaggaacctggcgacattttcacttactccgaattcgatggcatcctcggactggcgtacccgtccctggcttcacaatattctgtgcccgtctttgacaacatgatgaagcagcacctcgtggctcaagaccttttctctgtttatatgaatagccagggacagggttccatgctgatactgggggctattgacccctcatactacgtcggagcacttcattgggtccctgtgacggttcagcagtattggcagttcaccgtcgaccggataaccgtggatggagttgtcgtggcctgcgacggaggttgtcaagcgattcttgataccggcacatcactcctcgttgggccctcctctgatatattgaacatccagacggttatcggcgcaacccagggccaatacggggaattcgacattgattgcggttccctgagatcgaagccaacggtggtattcgagattaatggtcgcgaatacccgctcccgcccactgcttatactaatcaggaccagggcttctgcacttcgggttttcaaggagatgatagccagcaatgggttcttggcgacgtgtttatccgggaatactattccgtatttgatcgtgtcaacaacagggtcggcctggcaaaggccatt

SEQ ID NO: 14 - DNA, 1095 bp, Synthetic construct
  source 1..1095: mol_type = other DNA
tccatcatcagccgaatcccgcttcacaaaggaaagagtctgcggaaggcgctcaaagagcgcggacttctggaggactttttgaaaaaccatcagtacgctgtctcaaggaagcactcatcgtccgggaaggtcgcaagagagagccttaccaactacctggattgccagtacttcgggaagatatacattggcacaccgccccaagagttcaccgtggttttcgacactggcagttcagacctctgggtgccatctgtctactgcaattctgatgcctgtcagaatcatcatcgcttcgacccgtctaagtcgtccacatttcaaaacatggataagtcgctgtctatccaatatggtaccggttcgatgcagggtcttttgggctatgacacagttactgtctcaaatattgtcgatcgccaccagacggtcggcctgagcacccaagagcccggcgacgtgttcacttactctgaattcgatggcatcctcggactggcctacccctctctcgcgagcgaatactcggtaccagtgttcgataacatgatggataggcacctggtcgcccaagatctcttctccgtctatatgagtcggaacgagcagggttccatgttgactctcggggctatagatcgatcctattacacggggtcgttgcattggatcccagtgacggttcaggagtactggcagtttacggtcgatcgggttaccgtggacggcgtggtcgtagcctgtgatggcggatgccaggctattttggacaccggtaccagcatgctcgtgggaccgggttcagacattttcaatattcagcaagccatcggtgcaaccgagggacaatttggggagttcgacatcgattgcggcagacttagctctatgcccactgttgtctttgaaatcaacgggaaaaagtaccctctccctccttccgcttatacgagtcaggaccagggcttttgtacatcgggttttcaaggagattattcaagccagcaatggattcttggcgacgtattcatacgtgaatactattccgttttcgaccgcgcaagcaaccgtgttggattggcgaaagctatc

SEQ ID NO: 15 - DNA, 1101 bp, Synthetic construct
  source 1..1101: mol_type = other DNA
gcttttcgtcggatccctctgaccaagggaaaaacgctgcgcaaggttcttaaggagcatgggcttctggagagttttctcaagagccacaagtattctccttcttcgaaatatcagctttatggggaagcagctaaggtcaccgacgaacccctgacaaattacctcgactcgcaatactttggcaaaatctacattgggacaccaccgcaggaattcactgtcgtatttgataccggctcatccaacctttgggtgccgtctgtctactgcaattcggatgcgtgtcagaaccatcaccgattcaatccagcctcgtctactaccttccgatcaacccaggagccgctcagtattcagtatggcaccggctcaatggaaggcgtcctcggttacgatacagtcactgtcagtcaaattgttgtgcctgatcagatcttcggactctcgacccaagagcctggggagatcttcacgtactccgagttcgacgggatcctgggtctgggatacccctccttggcggaggatcaggccactccagttttcgacaatatgatgaacaaaaacctcgtggcccaagatctcttctctgtctatatgagcagggactcccagggttcgatgctgattcttggcgcgatagatccctcctattacacgggttcactgcattgggtgcccgttacggagcagggatattggcagttcagtgtcgattcaattaccgtcaacggccaggttgtggcttgtgaaggcggttgtcaagctatcttggacactggcacaagcctcctcgttggaccgagctacgatatcgccaacatccaatccattatcggagccacccagggccaatacggagaatatgacatcaactgcagcaacctgagcagtatgcccacggtggtagtccacataaacgggcgtcaatacccacttcctccctccgcgtatactaatcaggatcagggtttgtgctcctccggttttcaatctgagggctcagatcagttgtggatcttgggggacgtgtttataagagagtactactcggtgttcgaccggggaaacaatcgcgtcggtctggcaacggcagta

SEQ ID NO: 16 - DNA, 1095 bp, Synthetic construct
  source 1..1095: mol_type = other DNA
actggtattacccgcattcctctgcacaagggcaaaccattgcgcaaagcccttaaggaacacggcttgctcgaggatttcctgcaaaagcatcagagcgcagtctcgtccaagtatagctcttttggggaagtagcttcagagccgttgacgtcatacctggacagtcagtacttcggaaagatctacatcggaacgccgccccaggaattcaccgtcgttttcgacactggctcctcggatttgtgggtaccctctgtttactgcaaaagcgatgcatgccaaaatcatcagcgttttgatccatcaatgtcgagtacattccagaatctcgggaagcccttgtctatccaatacggcaccggatctatgcagggcttcctcggttatgataccgtcactgtgtccgacatagtcgatccacaacagactgtcggcctctccacacaagagcctggggatgtgttcacttactcggaatttgacggaatcttgggcctggcgtatccgagcctcgcttccgaatactccgtccccgttttcgataacatgatgaatcggcatctggttgctcaagacctcttctccgtctatatggatcgaaatggacaagagtcgatgcttacgctgggagctatcgacccgagctactatactggatctctccactgggtccctgtgacgcttcaaaagtactggcagtttaccgtcgacagtgtgacgatcggtggtgttgtcgttgcctgcgacggcggttgccaagcgattctggataccggcacctcaatgcttgtgggcccttcgagtgatattctgaacatccagatggcgataggggccacacagaaccaatatggggagttcgacattgactgtggttcgcttagttcgatgcccaccgtggtgttcgagatcaactcacgcatgtacccgctcacacctagcgcatatacaaatcaggaccagggtttctgtacaagcggttttcagggagaaaacaattcccagcaatggatccttggggacgtattcataagggagtattactctgtgtttgatcgggccaacaacagagtcggtctggctaaggccgtt

SEQ ID NO: 17 - protein, 323 aa, Bos taurus
GEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSDILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 18 - protein, 323 aa, Camelus dromedarius [sequence omitted]

(19) United States
(12) Patent Application Publication
(10) Pub. No.: US 2026/0000087 A1
(43) Pub. Date: Jan. 1, 2026

(54) Aspartic Protease, Methods and Uses Thereof

(71) Applicant: CHR. HANSEN A/S, Hoersholm (DK)

(72) Inventor: Christian Jaeckel, Boege (DK)

(73) Assignee: CHR. HANSEN A/S, Hoersholm (DK)

(21) Appl. No.: 18/848,499

(22) PCT Filed: Apr. 3, 2023
(86) PCT No.: PCT/EP2023/058636

§ 371 Date: Sep. 18, 2024

(30) Foreign Application Priority Data
Apr. 6, 2022 (EP) 22166960.9

(51) Int. Cl. A23C 19/032; A23C 19/068; A23C 19/072; C12N 9/64

(52) U.S. Cl. CPC A23C 19/032; A23C 19/0688; A23C 19/072; C12N 9/6478; C12N 9/6483; C12Y 304/23004

(57) ABSTRACT

The present invention generally relates to aspartic proteases (EC 3.4.23) for producing cheese.

### TECHNICAL FIELD

The present invention or disclosure generally relates to an aspartic protease, preferably a coagulant, more preferably a chymosin, for producing cheese with improved cheese yield, preferably with improved cheese yield and improved proteolysis. The cheese may be, for example, Cheddar cheese or Continental cheese.

### BACKGROUND

### Aspartic Proteases

Aspartic proteases (EC 3.4.23) are proteolytic enzymes, preferably peptidases, more preferably endopeptidases, having two aspartic acid residues in the active site that are relevant for their catalytic activity. These enzymes can be found, for example, in animals, plants or fungi and are relevant in the cheese-making industry as milk-clotting enzymes.

Enzymatic coagulation of milk by milk clotting enzymes or coagulants is one of the most important processes in the manufacture of cheeses. The enzymatic coagulation of milk by milk clotting enzymes or coagulants can be made by chymosins (EC 3.4.23.4), pepsins (EC 3.4.23.1), endothiapepsins (EC 3.4.23.22), or mucorpepsins (EC 3.4.23.23). For the sake of completeness, a chymosin is to be considered as a coagulant, however, not all coagulants are chymosins.

Chymosin (EC 3.4.23.4) and pepsin (EC 3.4.23.1) are milk clotting enzymes of the mammalian stomach. When produced in the gastric mucosal cells, chymosin and pepsin occur as enzymatically inactive pre-prochymosin and pre-pepsinogen, respectively. When pre-prochymosin is excreted, an N-terminal peptide fragment, the pre-fragment (signal peptide) is cleaved off to give prochymosin including a pro-fragment. Prochymosin is a substantially inactive form of the enzyme which, however, becomes activated under acidic conditions to the active chymosin by autocatalytic removal of the pro-fragment. This activation occurs in vivo in the gastric lumen under appropriate pH conditions or in vitro under acidic conditions.

Enzymatic milk coagulation is a two-phase process: a first phase where a proteolytic enzyme attacks K-casein, resulting in a metastable state of the casein micelle structure and a second phase, where the milk subsequently coagulates and forms a coagulum. Chymosin (EC 3.4.23.4) has a high specificity and predominantly clots milk by cleavage of a single 104-Ser-Phe-|-Met-Ala-107 bond in kappa-chain of casein. As a side-activity, chymosin also cleaves alpha-casein primarily between Phe23 and Phe24 and beta-casein primarily between Leu192 and Tyr193. The resulting peptides alpha_{S1}(1-23) and beta(193-209) are further degraded by proteases and peptidases from microbial cultures added to the ripening cheese.

The formation of the alpha_{S1}(1-23) peptide has been described to contribute to softening of the cheese texture (Creamer & Olsen, 1982). A correlation of both parameters has, for example, been found comparing chymosins from Bos taurus and Camelus dromedarius. While bovine chymosin cleaves alpha_{S1}-casein between Phe23 and Phe24 faster than camel chymosin, it yields softer cheeses with higher texture breakdown, e.g. Cheddar (Creamer & Olsen, 1982, Bansal et al. 2009) and mozzarella (Moynihan et al. 2014).

Access to cheese coagulants with a varying degree of alpha-casein or alpha_{S1}(1-23) peptide formation may enable the cheesemaker to impose different levels of softness to the cheese matrix. Therefore, a chymosin, preferably, a wild-type chymosin, with an increased alpha-casein or alpha_{S}1(1-23) peptide formation in cheese making is thus of high industrial interest. Coagulants with a fine-tuned alpha-casein or alpha_{S1}-casein proteolysis would facilitate the manufacturing of a wide variety of cheese types with optimal curd firmness, such as hard cheeses.

### Cheese Ripening

Cheese ripening (or cheese maturation) is a slow and expensive process that can go up to 2 years, depending on the cheese type. Costs associated to cheese ripening process come primarily from the inventory cost of delaying the sale of a large proportion of a year's production, the capital cost associated with ripening rooms and the need to control temperature and relative humidity.

Further, as a result of the extended ripening periods, the cheese has very low water content, develop strong taste and aroma, and are crumbly and dry. Industrial processing of hard cheeses can be very demanding, as it may require intense handwork for long periods, as well as good logistics (space) throughout the long ripening period. Parmigiano, Grana Padano, Pecorino, Cheddar, Gruyere, Emmental, and Mimolette are some of the most popular hard cheeses. Hence, methods to accelerate cheese ripening are highly relevant in the cheese making industry (McSweeney, P. L. H. 2007).

Finally, proteolysis is an important biochemical event during the ripening of most cheese varieties, with a major impact on flavor and texture. Proteolysis in cheese can be divided into three phases: proteolysis in milk before cheese manufacture, the enzymatically induced coagulation of the milk, and proteolysis during cheese ripening.

The profile of an optimal coagulant for cheese making differs between cheese types. The most common chymosins on the market are the bovine and camel chymosins, either genetically-engineered or not. The bovine chymosin is the chymosin most frequently used in the making of Cheddar cheese and similar hard cheeses, which may be sold under different names. Alternatively, a camel chymosin could be used for Cheddar cheese, for example, as it provides higher cheese yield and firmness compared to a bovine chymosin. However, camel chymosins are not used on the Cheddar market due to the low proteolytic activity of camel chymosin in cheese curd which undesirable extends the ripening time too much. Thus, a camel chymosin is less suitable for hard cheese manufacturing than a bovine chymosin as a result of its low proteolytic activity, both in milk coagulation and cheese ripening.

In conclusion, it is desirable to have an aspartic protease, preferably a coagulant, more preferably a chymosin, with low proteolytic activity in milk coagulation leading to a higher cheese yield, and with high proteolytic activity in cheese curd leading to faster ripening. The available art is silent about aspartic proteases contributing simultaneously to low proteolysis in milk coagulation for increased cheese yield and high proteolysis in the cheese curd for faster ripening. In other words, it is desirable to have an aspartic protease, preferably a coagulant, more preferably a chymosin with an improved specific clotting activity (C) with the proteolytic activity (P), or C/P ratio, over a bovine chymosin while simultaneously having a higher degradation of alpha-casein or alpha_{S1}-casein over a camel chymosin such that a higher cheese yield is obtained coupled to an appropriated proteolysis.

### SUMMARY

This invention or disclosure relates to an aspartic protease, preferably a coagulant, more preferably a chymosin, that combines low proteolysis in milk coagulation for increased cheese yield with high proteolysis in the cheese curd for faster ripening. In a preferred embodiment, the aspartic protease, or coagulant or chymosin is an wild-type aspartic protease, wild-type coagulant or wild-type chymosin. Some cheese types require fast ripening and consequently high proteolysis in the cheese curd. This is the case of Cheddar cheese, Continental cheese and similar cheeses.

The purpose of this invention or disclosure is to provide an aspartic protease or coagulant or chymosin for use in cheese making, in particular but not limited to Gouda cheese or Cheddar cheese, wherein the cheese yield is higher than the one currently obtained by the use of a bovine chymosin and wherein the degradation of intact alpha casein or alpha_{S1}-casein is higher than the one obtained by the use of a camel chymosin; preferably wherein the cheese yield is higher than the one currently obtained by the use of a bovine chymosin while the degradation of alpha-casein or alpha_{S1}-casein is at least the same as the one obtained by the use of a bovine chymosin; more preferably wherein the cheese yield and the degradation of alpha-casein or alpha_{S1}-casein are higher than the ones currently obtained by the use of a bovine chymosin.

The present invention or disclosure thereby solves the need for an aspartic protease or coagulant or chymosin with a suitable C/P ratio, preferably versus a bovine chymosin, while simultaneously improving cheese texture by breaking down more alpha_{S}1-casein than a camel chymosin, preferably more than a bovine chymosin. A preferred embodiment may be, for example, the combination of low proteolysis in milk coagulation for increased cheese yield and high proteolysis in cheese curd for fast ripening, as compared to a bovine chymosin.

The purpose of the invention or disclosure has been achieved by an aspartic protease herein disclosed. None of the known aspartic proteases, coagulants or chymosins (camel or bovine chymosins), wild-type or engineered, combines such properties. Thus, for the first time, it is disclosed an aspartic protease ideal for use in cheese making, such as hard cheese making, with ideal proteolysis and yield.

Therefore, this invention or disclosure relates to the following aspects: a DNA sequence encoding a peptide or polypeptide wherein the peptide or polypeptide has aspartic protease activity or chymosin activity or is an aspartic protease (EC 3.4.23) or is a chymosin (EC 3.4.23.4), wherein said peptide or polypeptide leads to a higher cheese yield over the one currently obtained by the use of a bovine chymosin and wherein the degradation of intact alpha casein or alpha_{S1}-casein is higher than the one obtained by the use of a camel chymosin; a vector comprising said DNA sequence; a host cell comprising said DNA sequence or said vector; a peptide or polypeptide encoded by the DNA sequence; a milk clotting composition comprising said peptide or polypeptide; a method for preparing a food product wherein the peptide or polypeptide or the milk composition is added to milk; and the use of the peptide or polypeptide as an aspartic protease or coagulant or chymosin.

### Definitions

All definitions of herein relevant terms are in accordance of what would be understood by the skilled person in relation to the herein relevant technical context.

The term “aspartic protease” relates to a proteolytic enzyme of the EC 3.4.23, preferably a peptidase, more preferably an endopeptidase, having two aspartic acid residues in the active site that are relevant for their catalytic activity. An aspartic protease is relevant in the cheese-making industry as milk-clotting enzymes.

The term “chymosin” relates to an enzyme of the EC 3.4.23.4 class. Chymosin has a high specificity and predominantly clots milk by cleavage of a single 104-Ser-Phe-|-Met-Ala-107 bond in kappa-chain of casein. As a side-activity, chymosin also cleaves alpha-casein primarily between Phe23 and Phe24 and beta-casein primarily between Leu192 and Tyr193. The resulting peptides alpha_{S}1(1-23) and beta(193-209) will be further degraded by proteases from microbial cultures added to the ripening cheese. A chymosin is relevant in the cheese-making industry as milk-clotting enzymes.

The term “chymosin activity” relates to chymosin activity of a chymosin enzyme as understood by the skilled person in the present context. The skilled person knows how to determine herein relevant chymosin activity.

As known in the art—chymosin specificity may be determined by the so-called C/P ratio, which is determined by dividing the specific clotting activity (C) with the proteolytic activity (P).

The detailed description provides an example of a standard method to determine specific chymosin activity—alternatively termed clotting activity or milk clotting activity. As an example, the specific clotting activity (C) may be determined using the REMCAT method, which is the standard method developed by the International Dairy Federation (IDF 157 or ISO 11815|IDF 157:2007).

The term “specific clotting activity” describes the milk clotting activity of a chymosin polypeptide and can be determined according to assays well known in the art. Therefore, “specific clotting activity” or “milk clotting activity” can be interchangeably used. A preferred method for determining the specific clotting activity in terms of IMCU/mg of protein is the standard method developed by the International Dairy Federation (IDF method), which comprises steps, wherein milk clotting activity is determined from the time needed for a visible flocculation of a milk substrate and the clotting time of a sample is compared to that of a reference standard having known milk-clotting activity and the same enzyme composition by IDF Standard 110B as the sample. Samples and reference standards are measured under identical chemical and physical conditions. The detailed description provides an example of a standard method to determine the “specific clotting activity” or the “milk clotting activity”. The detailed description also provides an example of a standard method to determine the proteolytical activity.

Alternatively, methods to determine the “specific clotting activity” and “proteolytic activity” can also be found in WO2013174840, WO2013164479, WO2015128417, WO2016207214, WO2017037092, WO2017198810 or WO2017198829 can also be used.

As known in the art—a higher C/P ratio implies generally that the loss of protein during e.g. cheese manufacturing due to non-specific protein degradation is reduced, i.e. the yield of cheese is improved.

It is also routine work for the skilled person to determine if a relevant peptide or polypeptide (e.g. camel or bovine wildtype chymosin or other) has chymosin activity or not.

The term “primary proteolysis” in cheese may refer to changes in beta-, gamma-, alpha_{S}-caseins, peptides, and other minor bands that are detected by polyacrylamide gel electrophoresis. Said changes are the result of the activity of the aspartic protease, coagulant or chymosin used. The term “alpha-cleavage” or “cleavage of alpha-casein” or “alpha-casein cleavage” means any enzymatic cleavage of alpha-casein. Generally, degradation of alpha-casein contributes to cheese texture softening. Details of a method of determining primary proteolysis and the “alpha-casein cleavage” are given in the detailed description below.

The term “alpha_{S1}-cleavage” or “cleavage of alpha_{S1}-casein” means any enzymatic cleavage of alpha_{S1}-casein, such as e.g. cleavage between Phe23 and Phe24, resulting in the formation of alpha_{S1}(1-23) peptide. Alpha_{S1}-cleavage may be determined by quantifying the alpha_{S1}-cleavage peptide 1-23 obtained by incubating skim milk or a cheese sample, as the case may be, with a reference aspartic protease, or a reference coagulant or a reference chymosin or with the aspartic proteases herein disclosed, wherein quantification is carried out by RP-HPLC coupled to an ESI-Q-TOF mass spectrometer. Details of a method of determining alpha_{S1}-casein cleavage are given in the detailed description below. Generally, degradation of alpha_{S1}-casein contributes to cheese texture softening.

The term “IMCU” means International Milk-Clotting Units. In the context of the present invention or disclosure “IMCU/L of milk” or IMCU/g_{protein in milk} correspond to the dosage or strength recommended for cheese making, while “IMCU/ml” corresponds to the average activity of a commercial coagulant or commercial composition of coagulants.

The term “mature polypeptide” means a peptide in its final form following translation and any post-translational modifications, such as N-terminal processing, C-terminal truncation, glycosylation, phosphorylation, etc. In the present context, a mature chymosin polypeptide is the active chymosin polypeptide sequence—i.e. without the pre-part and/or pro-part sequences.

The term “Sequence Identity” relates to the relatedness between two amino acid sequences or between two nucleotide sequences and may calculated according to the methods available to the person skilled in the art. For purposes of the present invention or disclosure, the degree of sequence identity between two amino acid sequences may be determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

The term “wild-type” chymosin peptide means a chymosin expressed by a naturally occurring organism, such as e.g. a mammalian found in nature.

The term “wild-type” peptide or polypeptide refers to a nucleotide sequence or peptide or polypeptide sequence as it occurs in nature, i.e. nucleotide sequence or peptide or polypeptide sequence which hasn't been subject to targeted mutations by the act of man.

In the present context, reference aspartic protease or a reference coagulant or a reference chymosin may be a commercially available.

In the present context a bovine chymosin is SEQ ID NO: 17 and a camel chymosin is SEQ ID NO: 18. A bovine chymosin may also be CHY-MAX®, CHY-MAX® Plus, CHY-MAX® Extra, all from Chr. Hansen A/S. A camel chymosin may also be CHY-MAX® M from Chr. Hansen A/S.

The term “food product” refers to a kind of milk-based product intended to be used as food, including, but not limited to, cheese, milk, skimmed milk, acidified milk, butter milk, condensed milk, spread, margarine, yoghurt, ice cream, milk powder, butter, dulce de leche, among others.

The term “dairy product” is intended to include any food product made using milk or milkbased product, including, but not limited to, cheese, milk, skimmed milk, acidified milk, butter milk, condensed milk, spread, margarine, yoghurt, ice cream, milk powder, butter, dulce de leche, among others.

The term “cheese” or “cheese product” are used interchangeably. The term “cheese” is understood to encompass any cheese, including, but not limited to, hard cheeses such as Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, enzyme-modified cheese (EMC) or cheese-like product such as vegan cheese or non-dairy cheese.

The term “processed cheese” is preferably manufactured from cheese or cheese analogues by cooking and emulsifying the cheese, such as with emulsifying salts (e.g. phosphates and citrate). The process may further include the addition of spices/condiments.

The term “enzyme-modified cheese” or “EMC” is understood as cheese curd which has been treated with enzymes to produce a concentrated cheese flavor ingredient which may have approximately 15-30 times the flavor intensity of natural cheese. EMCs are available as pastes or dried to form powders and are used to give a cheese flavor note to products such as processed cheese or analogue cheese, cheese powders, soups, sauces, dips, crackers, salad dressings and in coatings for snack foods.

The term “cheese-like product” is understood as cheese-like products which contain fat, such as e.g. milk fat (e.g. cream or butter) or vegetable oil, as a part of the composition, and which further contain, as part of the composition, one or more non-milk constituents, such as e.g. a vegetable constituent (e.g. vegetable protein or vegetable oil). In the present context, a “cheese-like product” includes a non-dairy cheese, also known as vegan cheese, wherein the fat used to make the non-dairy cheese is vegetable oil or an emulsion such as water-in-oil emulsion, instead of milk fat. In the present context, a cheese-lie product is a vegan cheese or non-dairy cheese.

In the present context MACY means moisture-adjusted cheese yield. This concept is well know in the art.

### BRIEF DESCRIPTION OF THE FIGURES

FIG. 1. Cheddar cheese yield corrected for dry matter (DM), where the black bar represents a bovine chymosin (reference C), the diagonal strap bar represents a camel chymosin (reference B), and the white bar represents an aspartic protease or coagulant or chymosin represented of SEQ ID NO: 1.

FIG. 2. Casein degradation (%) at 6-weeks of ripening in Cheddar cheese using a bovine chymosin (reference A), or an aspartic protease or coagulant or chymosin represented by SEQ ID NO: 1, 2 or 3.

FIG. 3. Content of intact alpha-casein (mg/g) in Cheddar cheese at 1-, 6- and 12-weeks of ripening using a bovine chymosin (reference A), or an aspartic protease or coagulant or chymosin represented by SEQ ID NO: 1, 2 or 3.

FIG. 4. Content of intact alpha-casein (mg/g) in Gouda Cheese at 1-, 2-, 4- and 9-weeks of ripening using a bovine chymosin (reference D), or an aspartic protease or coagulant or chymosin represented by SEQ ID NO: 1.

FIG. 5. Content of intact alpha-casein (mg/g) in Cheddar cheese #1 at 1-, 6- and 12-weeks of ripening using a bovine chymosin (reference D), or an aspartic protease or coagulant or chymosin represented by SEQ ID NO: 1.

FIG. 6. Content of intact alpha-casein (mg/g) in Cheddar cheese #2 at 1-, 6- and 12-weeks of ripening using a bovine chymosin (reference D), or an aspartic protease or coagulant or chymosin represented by SEQ ID NO: 1.

### DETAILED DESCRIPTION

### First Aspect

The first aspect relates to a DNA sequence comprising:
a nucleotide sequence having at least 75% sequence identity with SEQ ID NO: 9 or 13, preferably at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 9 or 13; or
a nucleotide sequence having at least 75% sequence identity with SEQ ID NO: 10 or 14, preferably at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 10 or 14; or
a nucleotide sequence having at least 75% sequence identity with SEQ ID NO: 11 or 15, preferably at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 11 or 15; or
a nucleotide sequence having at least 75% sequence identity with SEQ ID NO: 12 or 16, preferably at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 12 or 16.

Preferably, the DNA sequence encodes a peptide or a polypeptide such as an aspartic protease or a coagulant or a chymosin having a higher specific clotting activity (C)/proteolytic activity (P) ratio than a chymosin represented by SEQ ID NO: 17 or SEQ ID NO: 18.

Preferably, the DNA sequence encodes a peptide or a polypeptide such as an aspartic protease or a coagulant or a chymosin, wherein the peptide or polypeptide cleaves alpha-casein with a higher frequency than SEQ ID NO: 17 or SEQ ID NO: 18, meaning that less alpha-casein or intact alpha-casein is measured in a sample prepared with said peptide or polypeptide versus a sample prepared with SEQ ID NO: 17 or SEQ ID NO: 18.

Preferably, the DNA sequence encodes a peptide or a polypeptide such as an aspartic protease or a coagulant or a chymosin, wherein the peptide or polypeptide cleaves alpha_{S1}-casein with a higher frequency than SEQ ID NO: 17 or SEQ ID NO: 18, meaning that less alpha-casein or intact alpha-casein is measured in a sample prepared with said peptide or polypeptide versus a sample prepared with SEQ ID NO: 17 or SEQ ID NO: 18. More preferably wherein alpha_{S1}-casein cleavage is determined by quantifying alpha_{S1}-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

Preferably, the DNA sequence encodes a peptide or a polypeptide such as an aspartic protease or a coagulant or a chymosin; more preferably wherein said peptide or polypeptide has a higher specific clotting activity (C)/proteolytic activity (P) ratio than a chymosin represented by SEQ ID NO: 17 or SEQ ID NO: 18 and/or wherein the peptide or polypeptide cleaves alpha_{S1}-casein with a higher frequency than SEQ ID NO: 17 or SEQ ID NO: 18, preferably wherein alpha_{S1}-casein cleavage is determined by quantifying alpha_{S1}-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

Preferably, the DNA sequence may be an isolated DNA sequence or a recombinant DNA sequence or a synthetic DNA sequence.

Preferably, the DNA sequence may further encodes a signal peptide sequence and/or a linker sequence and/or a sequence encoding for a glucoamylase. More preferably, the DNA sequence may further encodes a linker sequence and/or a sequence encoding for a glucoamylase. Even more preferably, the DNA sequence may further encodes a linker and a sequence encoding for a glucoamylase.

### Second and Third Aspects

This invention or disclosure also relates to a vector comprising a DNA sequence as described in the first aspect of the invention.

Further, the invention or disclosure concerns a host cell, preferably a recombinant host cell, comprising a DNA sequence and/or comprising a vector, in either case as described above.

Preferably, the host cell may be selected from Aspergillus or Bacillus or Pichia; more preferably, Aspergillus niger or Bacillus subtilis or Pichia pastoria; even more preferably Aspergillus niger var. awamori.

### Fourth and Fifth Aspects

Additionally, the invention or disclosure relates to a peptide or polypeptide encoded by a DNA sequence as described in the first aspect, preferably wherein the peptide or polypeptide has aspartic protease activity or chymosin activity or is an aspartic protease (EC 3.4.23) or is a chymosin (EC 3.4.23.4).

Preferably, the peptide or polypeptide may have a higher specific clotting activity (C)/proteolytic activity (P) ratio than a chymosin represented by SEQ ID NO: 17 or SEQ ID NO: 18.

Preferably, the peptide or polypeptide may cleave alpha_{S1}-casein with a higher frequency than SEQ ID NO: 17 or SEQ ID NO: 18, in particular wherein alpha_{S1}-casein cleavage is determined by quantifying alpha_{S1}-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

Preferably, the peptide or polypeptide may have a higher specific clotting activity (C)/proteolytic activity (P) ratio than a chymosin represented by SEQ ID NO: 17 or SEQ ID NO: 18 and may cleave alpha-casein with a higher frequency than SEQ ID NO: 17 or SEQ ID NO: 18, meaning that less alpha-casein or intact alpha-casein is measured in a sample prepared with said peptide or polypeptide versus a sample prepared with SEQ ID NO: 17 or SEQ ID NO: 18.

Preferably, the peptide or polypeptide may have a higher specific clotting activity (C)/proteolytic activity (P) ratio than a chymosin represented by SEQ ID NO: 17 or SEQ ID NO: 18 and may cleave alpha_{S1}-casein with a higher frequency than SEQ ID NO: 17 or SEQ ID NO: 18, in particular wherein alpha_{S1}-casein cleavage is determined by quantifying alpha_{S1}-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

Preferably, the peptide or polypeptide herein disclosed may comprise:
an amino acid sequence having at least 70% sequence identity with SEQ ID NO: 1 or 5, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99%, or 100% sequence identity with SEQ ID NO: 1 or 5; or
an amino acid sequence having at least 70% sequence identity with SEQ ID NO: 2 or 6, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99%, or 100% sequence identity with SEQ ID NO: 2 or 6; or
an amino acid sequence having at least 70% sequence identity with SEQ ID NO: 3 or 7, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99%, or 100% sequence identity with SEQ ID NO: 3 or 7, or
an amino acid sequence having at least 70% sequence identity with SEQ ID NO: 4 or 8, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99%, or 100% sequence identity with SEQ ID NO: 4 or 8.

Additionally, the invention or disclosure concerns a milk clotting composition comprising a peptide or polypeptide as described above.

### Sixth Aspect

The invention or disclosure relates to a method for preparing a food product, wherein the method comprises the following steps:
adding a milk clotting effective amount of a peptide or polypeptide to milk, wherein peptide or polypeptide is as described above; or
adding a milk clotting effective amount of a milk clotting composition comprising a peptide or polypeptide as described above; and
carrying out appropriate steps for preparing the food product.

Preferably, the food product may be a dairy product selected from a cheese, a cheese product, a processed cheese, a cheese-like product such as vegan cheese or non-dairy cheese, a butter, a yogurt, a cream, and a seasoning.

Preferably, the food product or dairy product may be a cheese or a cheese product.

Preferably, the cheese or cheese product may be Cheddar cheese or Continental cheese or Gouda cheese.

Preferably, the cheese or cheese product may be Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, enzyme-modified cheese (EMC) or cheese-like product such as vegan cheese or non-dairy cheese.

Preferably, the milk used in the method herein disclosed may be cow's milk, camel's milk, buffalo milk, goat's milk, sheep's milk, and/or a mixture thereof.

Preferably, the method is carried out with a peptide or polypeptide as described above, wherein the peptide or polypeptide has a higher specific clotting activity (C)/proteolytic activity (P) ratio than a bovine chymosin, such as SEQ ID NO: 17 or SEQ ID NO: 18, preferably SEQ ID NO: 17; and has a higher degradation of alpha-casein or alphaS1-casein than a camel chymosin, such as SEQ ID NO: 17 or SEQ ID NO: 18, preferably SEQ ID NO: 18.

### Seventh Aspect

Finally, the invention or disclosure also relates to the use of
a peptide or polypeptide with an amino acid sequence having at least 70% sequence identity with SEQ ID NO: 1 or 5, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 1 or 5; or
a peptide or polypeptide with an amino acid sequence having at least 70% sequence identity with SEQ ID NO: 2 or 6, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 2 or 6; or
a peptide or polypeptide with an amino acid sequence having at least 70% sequence identity with SEQ ID NO: 3 or 7, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 3 or 7;
a peptide or polypeptide with an amino acid sequence having at least 70% sequence identity with SEQ ID NO: 4 or 8, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 4 or 8;
as an aspartic protease (EC 3.4.23) in the preparation of a food product.

Preferably, the aspartic protease (EC 3.4.23) is a coagulant; more preferably the aspartic protease (EC 3.4.23) is a chymosin (EC 3.4.23.4).

Preferably, the food product may be a dairy product.

Preferably, the dairy product may be selected from a cheese, a cheese product, a processed cheese, a cheese-like product such as vegan cheese or non-dairy cheese, a butter, a yogurt, a cream, and a seasoning; more preferably the cheese or cheese product is Cheddar cheese or Continental cheese or Gouda cheese.

Preferably, the cheese or cheese product may be Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, enzyme-modified cheese (EMC) or cheese-like product such as vegan cheese or non-dairy cheese.

Preferably, the peptide or polypeptide has a higher specific clotting activity (C)/proteolytic activity (P) ratio than SEQ ID NO: 17 and/or SEQ ID NO: 18, preferably higher than SEQ ID NO: 17; and/or wherein the peptide or polypeptide cleaves alpha-casein with a higher frequency than SEQ ID NO: 17 and/or SEQ ID NO: 18, preferably higher than SEQ ID NO: 18; and/or wherein the peptide or polypeptide cleaves alpha_{S1}-casein with a higher frequency than SEQ ID NO: 17 and/or SEQ ID NO: 18, preferably higher than SEQ ID NO: 18; and/or wherein the alpha_{S1}-casein cleavage is determined by quantifying alpha_{S1}-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

### Materials and Methods

### Determination of Milk Clotting Activity

Milk clotting activity may be determined using the REMCAT method, which is the standard method developed by the International Dairy Federation (IDF 157 or ISO 11815|IDF 157:2007).

Milk clotting activity is determined from the time needed for a visible flocculation of a standard milk substrate prepared from a low-heat, low fat milk powder with a calcium chloride solution of 0.5 g per liter (pH≈6.5). The clotting time of a rennet sample is compared to that of a reference standard having known milk-clotting activity and having the same enzyme composition by IDF Standard 110B as the sample. Samples and reference standards are measured under identical chemical and physical conditions. Variant samples are adjusted to approximately 3 IMCU/ml using an 84 mM acetic acid buffer pH 5.5. Hereafter, 200 μl enzyme preparation is added to 10 ml preheated milk (32° C.) in a glass test tube placed in a water bath, capable of maintaining a constant temperature of 32° C.±1° C. under constant stirring.

The total milk-clotting activity (strength) of a rennet is calculated in International Milk-Clotting Units (IMCU) per ml relative to a standard having the same enzyme composition as the sample according to the formula:

$$Strength in IMCU / ml = \frac{Sstandard \times Tstandard \times Dsample}{Dstandard \times Tsample}$$

$$Sstandard : The milk - clotting activity of the international reference standard for rennet .$$

$$Tstandard : Clotting time in seconds obtained for the standard dilution .$$

$$Dsample : Dilution factor for the sample$$

$$Dstandard : Dilution factor for the standard$$

$$Tsample : Clotting time in seconds obtained for the diluted rennet sample from addition of enzyme to time of flocculation .$$

### Determination of Total Protein Content

Total protein content may preferably be determined using the Pierce BCA Protein Assay Kit from Thermo Scientific following the instructions of the providers.

### Calculation of Specific Clotting Activity

Specific clotting activity (IMCU/mg total protein) was determined by dividing the clotting activity (IMCU/ml) by the total protein content (mg total protein per ml).

### Determination of Proteolytic Activity

General proteolytic activity was measured using fluorescently labeled Bodipy-FL casein as a substrate (EnzChek; Molecular Bioprobes, E6638). Casein derivatives heavily labeled with pH-insensitive green-fluorescent Bodipy-FL result in quenching of the conjugate's fluorescence. Protease catalyzed hydrolysis re-leases fluorescent Bodipy-FL. This method is very sensitive and essential when dealing with coagulants that have a low general proteolytical activity, which is the case of CHY-MAX® M from Chr. Hansen A/S used as a reference in several examples. A 0.04 mg/ml substrate solution was prepared in 0.2M phosphate buffer pH 6.5, containing 100 mM NaCl, 5% glycerol, and 0.1% Brij. The aspartic proteases herein disclosed as well as the reference chymosins were solved in 20 mM malonate buffer, containing 100 mM NaCl, 5% glycerol, and 0.1% Brij. Of both substrate and aspartic protease or coagulant or chymosin solutions, 20 μL were mixed in a black 384-well Corning flat bottom polystyrene microtitter plate and fluorescence was continuously recorded in a fluorometer at 32° C. for 10 hours. Slopes of the linear part of fluorescence change were used to determine general proteolytic activity. Identical steps were made for the reference chymosins.

### Determination of the C/P Ratio

The C/P ratio is calculated by dividing the clotting activity (C) with the proteolytic activity (P).

### Determination of Primary Proteolysis

Primary proteolysis was determined using LabChip® electrophoresis system, in particular the LabChip® HT Protein Express Assay (PN 760499) in combination with the LabChip® GXII Touch™ Protein Characterization System, both from PerkinElmer Inc. Briefly, a set of standards of isolated caseins at a known concentration was used to establish a calibration curve. Subsequently, corresponding caseins were identified and quantified in cheese extract obtained from a cheese sample prepared according to the Examples. The output obtained is a concentration of alpha-casein and beta-casein, both in mg/g of cheese. The summed concentrations of the casein types (alpha-casein and beta-casein) is referred to as total casein. In cheese, the total casein decreases over time due to degradation and a result the soluble nitrogen (SN)/total nitrogen (TN) (%) increases. As a consequence, the primary proteolysis also increases. Thus, casein quantification allows to evaluate the primary cheese proteolysis. If required, the degree of casein degradation can be expressed as a fraction of total protein (C_{Ndegradation} (%)). Generally, the degree of casein degradation acts in a similar way as the SN/TN (%), meaning that it increases as the proteolysis proceeds over time. Alternatively, primary proteolysis may also be determined by the Kjeldahl nitrogen analysis.

### Determination of Alpha_{S1}-Cleavage

Alpha_{S1}-cleavage is determined by quantifying the alpha_{S1}-cleavage peptide 1-23 obtained in cheese or cheese sample prepared with a chymosin, wherein quantification is carried out by RP-HPLC coupled to an ESI-Q-TOF mass spectrometer.

Chymosin mediated proteolysis of milk proteins was characterized by determining profiles of water soluble peptides extracted at pH 4.6. A culture free cheese model made in 96 well plates was used for the study. In brief, 750 μl skim milk from Øllingegård, Denmark added glucono-delta-lactone (GDL) and calcium chloride was aliquoted into the wells of a 96 deep well plate. After 10 min from addition of GDL to the milk, variants of chymosin were added to individual wells of the plate to a final activity of 0.05 IMCU/ml. The formed coagulum was cut after 30 min from addition of rennet by thoroughly stirring the coagulum with a pipette tip; a new tip was used for each well. Subsequently, the plate was left for another 60 min before curd and whey was separated by centrifugation of the plate for 10 min at 2500 g. The milk was kept at 30° C. during renneting, cutting and syneresis. Finally, whey was decanted from the plate and the pellet of rennet curd left in the plate was stored for 4 days at room temperature. Peptides were extracted by adding 500 μl of 0.5 M tri-sodium citrate to each well and gentle shaking the plate for 24 hours at 37° C. The now fully dissolved rennet curd was then precipitated by adding hydrochloric acid to a final pH of 4.4-4.5. The plate was spun down in a centrifuge and the supernatant recovered for further analysis of pH 4.5 soluble peptides. Profiles of pH 4.5 soluble peptides were determined using RP-HPLC coupled to an ESI-Q-TOF mass spectrometer. The analysis was performed by using a liquid chromatography system (Agilent 1290 infinity, Agilent Technologies A/S, Santa Clara, California, USA) coupled to a mass spectrometer (G6540A Q-TOF, Agilent Technologies A/S, Santa Clara, California, USA). The column in the LC system was Ascentis Express Peptide ES-C18m, 2.7 μm, 100×2.1 mm (Supelco, Sigma-Aldrich, St. Louis, USA). The mobile phase consisted of eluent A (0.1% formic acid in water) and eluent B (Acetonitrile: 0.1% formic acid in water, 9:1). After equilibration of the column with 2% B, a sample volume of 10 μL was injected. The peptides were separated by gradient elution generated by increasing eluent B from 2% to 50% over 15 column volumes. The flow rate was 0.44 mL/min. Peptides were detected by continuously measuring the UV absorbance at 214 nm. By running MS scans from 100 to 2000 m/z the mass spectra were collected. MS/MS analysis was performed on the two most intense ions from each scan. A mix sample consisting of equal volume of all samples analyzed was prepared and this sample was analyzed for each sample. MS data were converted from the Agilent .d format to .mzml files using MSConvert ver. 3.0.6618. All further data analysis was done using R 3.1.3. Peptides were identified from MS/MS spectra using R package ‘MSGFplus’ version 1.05. Search database for peptide identification were limited to the bovine milk proteins: alpha_{S}1-casein, beta-casein, kappa-casein, beta-lactoglobulin, alpha-lactalbumin, lactoperoxidase and lactoferrin. Serine phosphorylation and methionine oxidation were included as variable modifications. R package ‘xcms’ v. 1.42.0 was used for detecting and grouping peaks across samples in a sampleset according to Smith et al. (2006). Massifquant method was used for peak detection and grouping of peaks was based on the density method. Identity was assigned to grouped peaks resulting in quantitative tables of approximately 200 identified peptides including alpha_{S1}-casein (1-23).

### EXAMPLES

### Example 1

A nucleotide sequence encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 was integrated into a targeted locus of an expression host such as Aspergillus niger or Aspergillus niger var. awamori. Preferably, a nucleotide sequence encoding for SEQ ID NO: 5 or SEQ ID NO: 6 or SEQ ID NO: 7 or SEQ ID NO: 8 was integrated into a targeted locus of an expression host such as Aspergillus niger or Aspergillus niger var. awamori. Alternatively, other expression hosts may be used such as Bacillus or Pichia, preferably Bacillus subtilis or Pichia pastoria.

Codon-optimized genes encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 may or not be used. Said genes/sequences may be SEQ ID NO: 9 or SEQ ID NO: 10 or SEQ ID NO: 11 or SEQ ID NO: 12 or SEQ ID NO: 13 or SEQ ID NO: 14 or SEQ ID NO: 15 or SEQ ID NO: 16. The skilled person is aware of how to prepare said codon-optimized genes, such that the expression of the peptide or polypeptide, in a host organism, is optimized.

The produced proteins were purified and characterized regarding proteolytic specificity for milk coagulation (C/P), as well as the degradation of intact alpha_{S1} casein in cheese curd.

Aspartic proteases represented by SEQ ID NO: 1-4 were independently used in cheese making. All values were normalized to the respective numbers obtained for a bovine chymosin, such as CHY-MAX® Plus from Chr. Hansen A/S, herein labeled as reference A. A camel chymosin (CHY-MAX® M from Chr. Hansen A/S) is herein labeled as reference B.

TABLE 1
Values are given in % of reference A.

| | | Degradation of intact |
|---|---|---|
| | C/P | alpha_{S1}-casein |
| Reference A | 100 | 100 |
| Reference B | 659 | 38 |
| SEQ ID NO: 1 | 176 | 135 |
| SEQ ID NO: 2 | 583 | 71 |
| SEQ ID NO: 3 | 513 | 204 |
| SEQ ID NO: 4 | 9444 | 105 |

SEQ ID NOS: 1-4 show a higher C/P ratio as compared to reference A (a bovine chymosin) and a higher degradation pattern of intact alpha_{S1}-casein as compared to reference B (a camel chymosin). Additionally, SEQ ID NOs: 1, 3 and 4 also have a higher degradation pattern as compared to reference A.

Based on the higher C/P ratios, the sequences herein disclosed are suitable aspartic proteases or coagulants for producing cheese with improved cheese yield, preferably with improved cheese yield and improved proteolysis.

### Example 2

Cheese, in particular Cheddar cheese, was produced. The cheese-making process is standard in the art. The coagulants used in this process were:
a camel chymosin (CHY-MAX® M from Chr. Hansen A/S) —reference B;
a bovine chymosin (CHY-MAX® from Chr. Hansen A/S) —reference C;
coagulant represented by SEQ ID NO: 1—coagulant 1.

Each coagulant was added in a dosage of 5300 IMCU/100 kg of milk (approximately 55 IMCU/L of milk).

### Cheese Yield

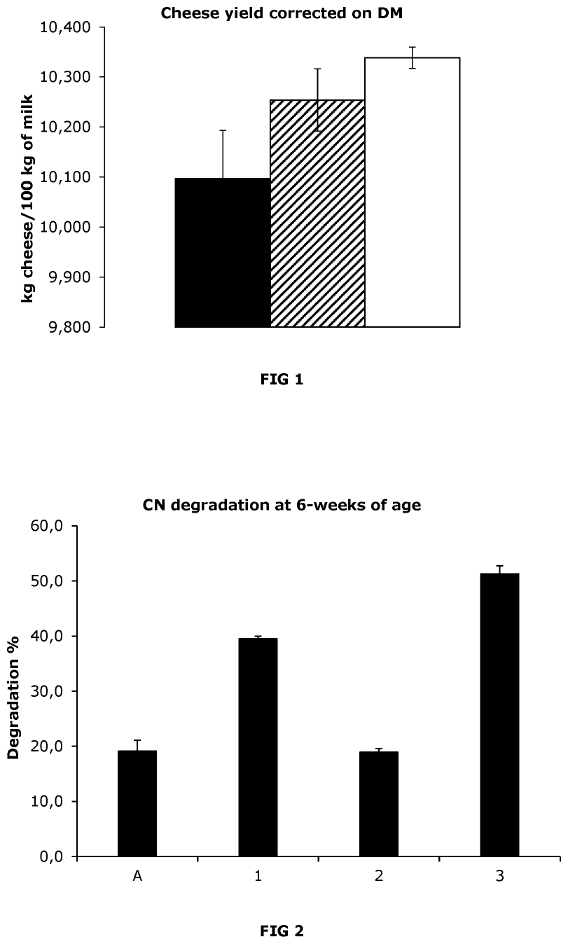

Cheese yield corrected on dry matter (DM) was calculated by weighting cheeses after demolding according to milk mass used. The gross yield called economical cheese yield (ECY) was adjusted by measuring the dry matter (DM) after 2-weeks of ripening. The economical cheese yield was then adjusted using dry matter as follows: ECY×DM of trial %/DM of reference %. Briefly, the yield adjusted on moisture was then used in order to remove the water retention effect on cheese yield. The average and standard deviation were calculated and plotted in FIG. 1. FIG. 1 shows an increase in cheese yield when cheese is made with coagulant 1 versus when cheese is made with a reference chymosin (either a camel chymosin or a bovine chymosin).

Further, to take into account the composition of the milk used, in terms of fat and protein, the cheese yield was further expressed by the adjusted yield (MACY) (moisture adjusted yield) divided by milk composition as the milk used may not be the same between different trials.

TABLE 2
Cheese yield determined by MACY divided by the milk composition, in
particular divided by the sum % of fat and protein in the milk.

| | |
|---|---|
| SEQ ID NO: 1 vs. reference B | +0.4 |
| Relative % diff between reference and SEQ ID NO: 1 | |
| SEQ ID NO: 1 vs. reference C | +0.6 |
| Relative % diff between reference and SEQ ID NO: 1 | |

### Primary Proteolysis

The primary proteolysis of cheeses produced by using coagulants 1, B and C was also determined and evaluated—Table 3.

TABLE 3
Proteolysis SN/TN (%) at 4-, 12- and 24-weeks, wherein SN represents
the soluble nitrogen and TN represents the total nitrogen.

| | Cheese ID | 4-weeks | 12-weeks | 24-weeks |
|---|---|---|---|---|
| Reference B | B.1 | 8.0 | 13.0 | 18.9 |
| | B.2 | 7.5 | 13.3 | 18.6 |
| Reference C | C.1 | 10.4 | 16.9 | 22.4 |
| | C.2 | 10.6 | 16.7 | 23.3 |
| Coagulant 1 | 1.1 | 13.1 | 22.5 | 28.0 |
| | 1.2 | 12.1 | 21.6 | 27.0 |

Table 3 shows that it is possible to modulate the primary proteolysis (ST/TN) according to the coagulant used. In particular Table 3 shows that coagulant 1 promotes a faster ripening process than references B and C regardless of the ripening period. Preferably a suitable primary proteolysis is obtained within a 4-week ripening period versus the respective levels obtained when references B and C are used. A value of at least 10%, preferably at least 13% is an acceptable value of proteolysis (ST/TN) at 4-weeks of ripening (approximately 1-month of ripening). Further, an optimal primary proteolysis is obtained within a 12-week ripening period versus the respective levels obtained when references B and C are used. A value of at least 20%, is an acceptable value of proteolysis (ST/TN) at 12-weeks of ripening (approximately 3-month of ripening). Identical results are expected for other types of cheese, such as Continental cheese or Swiss cheese type.

In conclusion, Example 2 shows that the aspartic proteases herein disclosed, in particular coagulant 1, lead to an increase of cheese yield while simultaneously providing the same or more primary proteolysis versus when a bovine or camel chymosin is used. In particular, coagulant 1 leads to an increase of cheese yield and to an increase of primary proteolysis versus when a bovine chymosin is used.

Based on Example 1, Table 1, identical results are expected for aspartic proteases, preferably coagulants, more preferably chymosins, represented by SEQ ID NO: 2-4.

### Example 3

Cheddar cheese was produced using a dosage of coagulant of 36 IMCU/L of milk. The coagulants tested were:
a bovine chymosin (CHY-MAX® Plus) —reference A;
coagulant represented by SEQ ID NO: 1—coagulant 1;
coagulant represented by SEQ ID NO: 2—coagulant 2;
coagulant represented by SEQ ID NO: 3—coagulant 3.

### Primary Proteolysis

The casein degradation profile was analyzed using electrophoresis (or LabChip® method) as described above, after about 6- and 12-weeks. In particular, alpha-casein degradation was also analyzed. Alpha-casein degradation plays a role in texture contributing to an increase of softness.

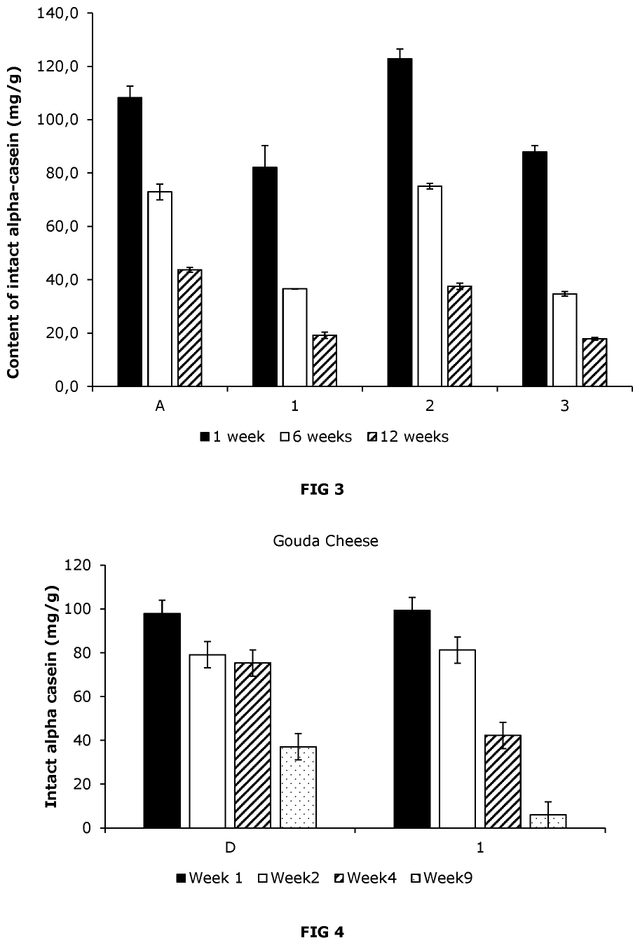

Coagulants 1 to 3 have at least similar (coagulant 2) or higher (coagulants 1 and 3) casein breakdown than the reference coagulant—FIG. 2. Further, coagulants 1 to 3 have at least the same (coagulant 2) or less (coagulants 1 and 3) intact alpha-casein than reference C, which indicates a better texture of cheese obtained with either coagulants 1 to 3 than with the reference chymosin used—FIG. 3. A lower amount of intact alpha-casein is representative of a higher degradation of intact alpha-casein, in particular versus the reference chymosin used.

Based on Table 1, identical results are expected for an aspartic protease, preferably coagulant, more preferably chymosin, represented by SEQ ID NO: 4.

### Example 4

Cheese, Gouda or Cheddar cheese, was also produced using:
a bovine chymosin (CHY-MAX® Extra from Chr. Hansen A/S) —reference D; and
a coagulant represented by SEQ ID NO: 1—coagulant 1.

TABLE 4

| | Dosage of | Milk | Cutting |
|---|---|---|---|
| Cheese | chymosin/coagulant | composition | point (Pa) |
| Continental | 36 IMCU/g_{protein in milk} | 3.4% fat | 130 |
| Gouda | | 3.6% protein | |
| Cheddar #1 | 36 IMCU/Lₘᵢₗₖ | 4% fat | 170 |
| (UK recipe) | | 3.6% protein | |
| Cheddar #2 | 61 IMCU/Lₘᵢₗₖ | 5.3% fat | 200 |
| (US recipe) | | 4.2% protein | |

### Cheese Yield

Cheese yield was determined as in Example 2—Table 5.

TABLE 5
Cheese yield determined by MACY divided by the milk composition, in
particular divided by the sum % of fat and protein in the milk.

| | SEQ ID NO: 1 vs. reference D |
|---|---|
| | Relative % diff between reference |
| Cheese | and SEQ ID NO: 1 |
| Continental Gouda | +0.7 |
| Cheddar #1 (UK recipe) | +0.5 |
| Cheddar #2 (US recipe) | +0.4 |

### Primary Proteolysis

The cheese yield obtained when SEQ ID NO: 1 used is used in the cheese-making process is higher than the cheese yield when a bovine chymosin is used, regardless of the cheese type or recipe used. This is in line with Example 1.

Further, alpha-casein of cheese was determined for each cheese—FIGS. 4-6. FIG. 4 shows that alpha-casein is degraded faster when coagulant 1 is used. In particular, when coagulant 1 is used, the alpha-casein fraction is significantly less at 4-weeks (about 40% remains in the cheese) and 9-weeks (about 5% remains in the cheese) of ripening versus then a reference chymosin is used as a coagulant.

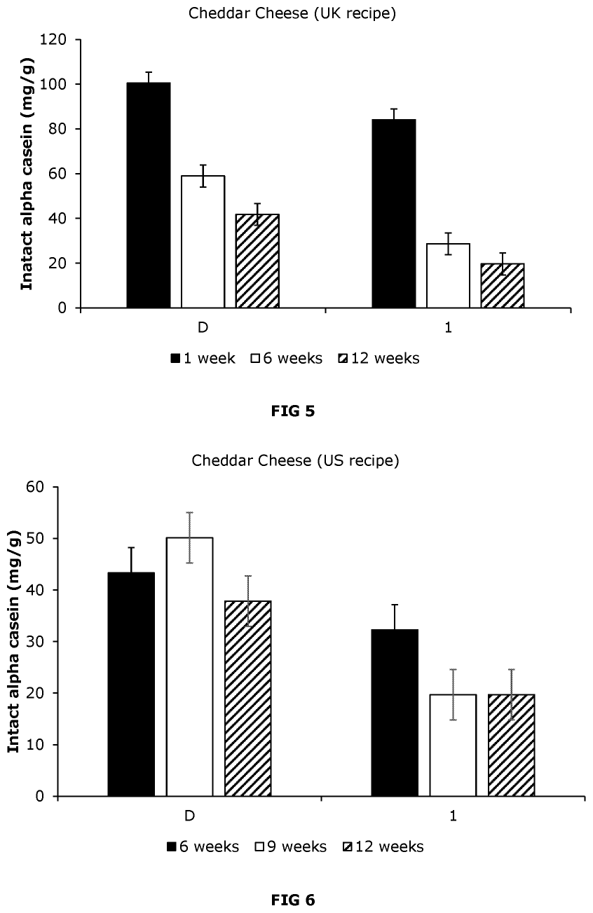

The ability to decrease the intact alpha-casein faster and therefore reduce the ripening time of a cheese while simultaneously contributing to improve softness is also appreciated in FIGS. 5-6. These figures confirm a faster alpha-casein breakdown for the cheese prepared with a coagulant represented by SEQ ID NO: 1 versus one prepared with a commercial reference. Thus, a faster alpha-casein cleavage is coupled with an improved softness of the cheese.

The purpose of this invention or disclosure is to provide an aspartic protease or coagulant or chymosin, preferably a wild-type aspartic protease or coagulant or chymosin, for use in food, such as for use in cheese making, where the cheese yield is higher than the one currently obtained by the use of a bovine chymosin or camel chymosin and wherein the degradation of intact alpha-casein or alpha_{S1}-casein is higher (less intact alpha-casein or less intact alpha_{S1}-casein is measured) than the one obtained by the use of a camel chymosin; preferably wherein the cheese yield is higher than the one currently obtained by the use of a bovine chymosin while the degradation of alpha-casein is at least the same as the one obtained by the use of a bovine chymosin; more preferably wherein the cheese yield and the degradation of alpha-casein or alpha_{S1}-casein are higher than the ones currently obtained by the use of a bovine chymosin. An aspartic protease as herein disclosed have a suitable C/P ratio, preferably versus a bovine chymosin, while simultaneously improving cheese texture by breaking down more alpha-casein or alpha_{S1}-casein than a camel chymosin, preferably more than a bovine chymosin.

SEQUENCE LISTING

SEQ ID NO: 1
GKIAREPLTNYLDTEYFGKIQIGSPPQEFTVVFDTGSSDLWVPSVFCNSDACQNHHRFNPSKSSTFQNMNRPLSIQYGTGSMQGFL
GYDTVTVSNIVVPHQTVGLSTQEPGDIFTYSEFDGILGLAYPSLASQYSVPVFDNMMKQHLVAQDLFSVYMNSQGQGSMLILGAID
PSYYVGALHWVPVTVQQYWQFTVDRITVDGVVVACDGGCQAILDTGTSLLVGPSSDILNIQTVIGATQGQYGEFDIDCGSLRSKPT
VVFEINGREYPLPPTAYTNQDQGFCTSGFQGDDSQQWVLGDVFIREYYSVFDRVNNRVGLAKAI
SEQ ID NO: 2
GKVARESLTNYLDCQYFGKIYIGTPPQEFTVVFDTGSSDLWVPSVYCNSDACQNHHRFDPSKSSTFQNMDKSLSIQYGTGSMQGLL
GYDTVTVSNIVDRHQTVGLSTQEPGDVFTYSEFDGILGLAYPSLASEYSVPVFDNMMDRHLVAQDLFSVYMSRNEQGSMLTLGAID
RSYYTGSLHWIPVTVQEYWQFTVDRVTVDGVVVACDGGCQAILDTGTSMLVGPGSDIFNIQQAIGATEGQFGEFDIDCGRLSSMPT
VVFEINGKKYPLPPSAYTSQDQGFCTSGFQGDYSSQQWILGDVFIREYYSVFDRASNRVGLAKAI
SEQ ID NO: 3
GEAAKVTDEPLTNYLDSQYFGKIYIGTPPQEFTVVFDTGSSNLWVPSVYCNSDACQNHHRFNPASSTTFRSTQEPLSIQYGTGSME
GVLGYDTVTVSQIVVPDQIFGLSTQEPGEIFTYSEFDGILGLGYPSLAEDQATPVFDNMMNKNLVAQDLFSVYMSRDSQGSMLILG
AIDPSYYTGSLHWVPVTEQGYWQFSVDSITVNGQVVACEGGCQAILDTGTSLLVGPSYDIANIQSIIGATQGQYGEYDINCSNLSS
MPTVVVHINGRQYPLPPSAYTNQDQGLCSSGFQSEGSDQLWILGDVFIREYYSVFDRGNNRVGLATAV
SEQ ID NO: 4
GEVASEPLTSYLDSQYFGKIYIGTPPQEFTVVFDTGSSDLWVPSVYCKSDACQNHQRFDPSMSSTFQNLGKPLSIQYGTGSMQGFL
GYDTVTVSDIVDPQQTVGLSTQEPGDVFTYSEFDGILGLAYPSLASEYSVPVFDNMMNRHLVAQDLFSVYMDRNGQESMLTLGAID
PSYYTGSLHWVPVTLQKYWQFTVDSVTIGGVVVACDGGCQAILDTGTSMLVGPSSDILNIQMAIGATQNQYGEFDIDCGSLSSMPT
VVFEINSRMYPLTPSAYTNQDQGFCTSGFQGENNSQQWILGDVFIREYYSVFDRANNRVGLAKAV
SEQ ID NO: 5
SGITRVPLLKGKSLRKSLKEHGLLEEFLQTHARKKLAHSGKIAREPLTNYLDTEYFGKIQIGSPPQEFTVVFDTGSSDLWVPSVFC
NSDACQNHHRFNPSKSSTFQNMNRPLSIQYGTGSMQGFLGYDTVTVSNIVVPHQTVGLSTQEPGDIFTYSEFDGILGLAYPSLASQ
YSVPVFDNMMKQHLVAQDLFSVYMNSQGQGSMLILGAIDPSYYVGALHWVPVTVQQYWQFTVDRITVDGVVVACDGGCQAILDTGT
SLLVGPSSDILNIQTVIGATQGQYGEFDIDCGSLRSKPTVVFEINGREYPLPPTAYTNQDQGFCTSGFQGDDSQQWVLGDVFIREY
YSVFDRVNNRVGLAKAI
SEQ ID NO: 6
SIISRIPLHKGKSLRKALKERGLLEDFLKNHQYAVSRKHSSSGKVARESLTNYLDCQYFGKIYIGTPPQEFTVVFDTGSSDLWVPS
VYCNSDACQNHHRFDPSKSSTFQNMDKSLSIQYGTGSMQGLLGYDTVTVSNIVDRHQTVGLSTQEPGDVFTYSEFDGILGLAYPSL
ASEYSVPVFDNMMDRHLVAQDLFSVYMSRNEQGSMLTLGAIDRSYYTGSLHWIPVTVQEYWQFTVDRVTVDGVVVACDGGCQAILD
TGTSMLVGPGSDIFNIQQAIGATEGQFGEFDIDCGRLSSMPTVVFEINGKKYPLPPSAYTSQDQGFCTSGFQGDYSSQQWILGDVF
IREYYSVFDRASNRVGLAKAI
SEQ ID NO: 7
AFRRIPLTKGKTLRKVLKEHGLLESFLKSHKYSPSSKYQLYGEAAKVTDEPLTNYLDSQYFGKIYIGTPPQEFTVVFDTGSSNLWV
PSVYCNSDACQNHHRFNPASSTTFRSTQEPLSIQYGTGSMEGVLGYDTVTVSQIVVPDQIFGLSTQEPGEIFTYSEFDGILGLGYP
SLAEDQATPVFDNMMNKNLVAQDLFSVYMSRDSQGSMLILGAIDPSYYTGSLHWVPVTEQGYWQFSVDSITVNGQVVACEGGCQAI
LDTGTSLLVGPSYDIANIQSIIGATQGQYGEYDINCSNLSSMPTVVVHINGRQYPLPPSAYTNQDQGLCSSGFQSEGSDQLWILGD
VFIREYYSVFDRGNNRVGLATAV
SEQ ID NO: 8
TGITRIPLHKGKPLRKALKEHGLLEDFLQKHQSAVSSKYSSFGEVASEPLTSYLDSQYFGKIYIGTPPQEFTVVFDTGSSDLWVPS
VYCKSDACQNHQRFDPSMSSTFQNLGKPLSIQYGTGSMQGFLGYDTVTVSDIVDPQQTVGLSTQEPGDVFTYSEFDGILGLAYPSL
ASEYSVPVFDNMMNRHLVAQDLFSVYMDRNGQESMLTLGAIDPSYYTGSLHWVPVTLQKYWQFTVDSVTIGGVVVACDGGCQAILD
TGTSMLVGPSSDILNIQMAIGATQNQYGEFDIDCGSLSSMPTVVFEINSRMYPLTPSAYTNQDQGFCTSGFQGENNSQQWILGDVF
IREYYSVFDRANNRVGLAKAV
SEQ ID NO: 9
GGGAAGATCGCGCGAGAGCCGCTCACAAATTATTTGGACACGGAGTACTTCGGCAAAATCCAGATCGGTAGCCCGCCTCAAGAGTT
TACAGTCGTGTTCGACACCGGTTCTTCGGATCTCTGGGTTCCTTCCGTCTTTTGCAATAGTGATGCCTGTCAGAACCATCATCGCT
TTAATCCATCCAAGTCTAGCACCTTCCAAAACATGAACCGCCCCCTGAGTATCCAATACGGCACCGGTAGTATGCAGGGGTTCTTG
GGTTATGATACAGTCACCGTATCGAACATCGTGGTTCCACACCAAACCGTAGGATTGAGCACTCAGGAACCTGGCGACATTTTCAC
TTACTCCGAATTCGATGGCATCCTCGGACTGGCGTACCCGTCCCTGGCTTCACAATATTCTGTGCCCGTCTTTGACAACATGATGA
AGCAGCACCTCGTGGCTCAAGACCTTTTCTCTGTTTATATGAATAGCCAGGGACAGGGTTCCATGCTGATACTGGGGGCTATTGAC
CCCTCATACTACGTCGGAGCACTTCATTGGGTCCCTGTGACGGTTCAGCAGTATTGGCAGTTCACCGTCGACCGGATAACCGTGGA
TGGAGTTGTCGTGGCCTGCGACGGAGGTTGTCAAGCGATTCTTGATACCGGCACATCACTCCTCGTTGGGCCCTCCTCTGATATAT
TGAACATCCAGACGGTTATCGGCGCAACCCAGGGCCAATACGGGGAATTCGACATTGATTGCGGTTCCCTGAGATCGAAGCCAACG
GTGGTATTCGAGATTAATGGTCGCGAATACCCGCTCCCGCCCACTGCTTATACTAATCAGGACCAGGGCTTCTGCACTTCGGGTTT
TCAAGGAGATGATAGCCAGCAATGGGTTCTTGGCGACGTGTTTATCCGGGAATACTATTCCGTATTTGATCGTGTCAACAACAGGG
TCGGCCTGGCAAAGGCCATT
SEQ ID NO: 10
GGGAAGGTCGCAAGAGAGAGCCTTACCAACTACCTGGATTGCCAGTACTTCGGGAAGATATACATTGGCACACCGCCCCAAGAGTT
CACCGTGGTTTTCGACACTGGCAGTTCAGACCTCTGGGTGCCATCTGTCTACTGCAATTCTGATGCCTGTCAGAATCATCATCGCT
TCGACCCGTCTAAGTCGTCCACATTTCAAAACATGGATAAGTCGCTGTCTATCCAATATGGTACCGGTTCGATGCAGGGTCTTTTG
GGCTATGACACAGTTACTGTCTCAAATATTGTCGATCGCCACCAGACGGTCGGCCTGAGCACCCAAGAGCCCGGCGACGTGTTCAC
TTACTCTGAATTCGATGGCATCCTCGGACTGGCCTACCCCTCTCTCGCGAGCGAATACTCGGTACCAGTGTTCGATAACATGATGG
ATAGGCACCTGGTCGCCCAAGATCTCTTCTCCGTCTATATGAGTCGGAACGAGCAGGGTTCCATGTTGACTCTCGGGGCTATAGAT
CGATCCTATTACACGGGGTCGTTGCATTGGATCCCAGTGACGGTTCAGGAGTACTGGCAGTTTACGGTCGATCGGGTTACCGTGGA
CGGCGTGGTCGTAGCCTGTGATGGCGGATGCCAGGCTATTTTGGACACCGGTACCAGCATGCTCGTGGGACCGGGTTCAGACATTT
TCAATATTCAGCAAGCCATCGGTGCAACCGAGGGACAATTTGGGGAGTTCGACATCGATTGCGGCAGACTTAGCTCTATGCCCACT
GTTGTCTTTGAAATCAACGGGAAAAAGTACCCTCTCCCTCCTTCCGCTTATACGAGTCAGGACCAGGGCTTTTGTACATCGGGTTT
TCAAGGAGATTATTCAAGCCAGCAATGGATTCTTGGCGACGTATTCATACGTGAATACTATTCCGTTTTCGACCGCGCAAGCAACC
GTGTTGGATTGGCGAAAGCTATC
SEQ ID NO: 11
GGGGAAGCAGCTAAGGTCACCGACGAACCCCTGACAAATTACCTCGACTCGCAATACTTTGGCAAAATCTACATTGGGACACCACC
GCAGGAATTCACTGTCGTATTTGATACCGGCTCATCCAACCTTTGGGTGCCGTCTGTCTACTGCAATTCGGATGCGTGTCAGAACC
ATCACCGATTCAATCCAGCCTCGTCTACTACCTTCCGATCAACCCAGGAGCCGCTCAGTATTCAGTATGGCACCGGCTCAATGGAA
GGCGTCCTCGGTTACGATACAGTCACTGTCAGTCAAATTGTTGTGCCTGATCAGATCTTCGGACTCTCGACCCAAGAGCCTGGGGA
GATCTTCACGTACTCCGAGTTCGACGGGATCCTGGGTCTGGGATACCCCTCCTTGGCGGAGGATCAGGCCACTCCAGTTTTCGACA
ATATGATGAACAAAAACCTCGTGGCCCAAGATCTCTTCTCTGTCTATATGAGCAGGGACTCCCAGGGTTCGATGCTGATTCTTGGC
GCGATAGATCCCTCCTATTACACGGGTTCACTGCATTGGGTGCCCGTTACGGAGCAGGGATATTGGCAGTTCAGTGTCGATTCAAT
TACCGTCAACGGCCAGGTTGTGGCTTGTGAAGGCGGTTGTCAAGCTATCTTGGACACTGGCACAAGCCTCCTCGTTGGACCGAGCT
ACGATATCGCCAACATCCAATCCATTATCGGAGCCACCCAGGGCCAATACGGAGAATATGACATCAACTGCAGCAACCTGAGCAGT
ATGCCCACGGTGGTAGTCCACATAAACGGGCGTCAATACCCACTTCCTCCCTCCGCGTATACTAATCAGGATCAGGGTTTGTGCTC
CTCCGGTTTTCAATCTGAGGGCTCAGATCAGTTGTGGATCTTGGGGGACGTGTTTATAAGAGAGTACTACTCGGTGTTCGACCGGG
GAAACAATCGCGTCGGTCTGGCAACGGCAGTA
SEQ ID NO: 12
GGGGAAGTAGCTTCAGAGCCGTTGACGTCATACCTGGACAGTCAGTACTTCGGAAAGATCTACATCGGAACGCCGCCCCAGGAATT
CACCGTCGTTTTCGACACTGGCTCCTCGGATTTGTGGGTACCCTCTGTTTACTGCAAAAGCGATGCATGCCAAAATCATCAGCGTT
TTGATCCATCAATGTCGAGTACATTCCAGAATCTCGGGAAGCCCTTGTCTATCCAATACGGCACCGGATCTATGCAGGGCTTCCTC
GGTTATGATACCGTCACTGTGTCCGACATAGTCGATCCACAACAGACTGTCGGCCTCTCCACACAAGAGCCTGGGGATGTGTTCAC
TTACTCGGAATTTGACGGAATCTTGGGCCTGGCGTATCCGAGCCTCGCTTCCGAATACTCCGTCCCCGTTTTCGATAACATGATGA
ATCGGCATCTGGTTGCTCAAGACCTCTTCTCCGTCTATATGGATCGAAATGGACAAGAGTCGATGCTTACGCTGGGAGCTATCGAC
CCGAGCTACTATACTGGATCTCTCCACTGGGTCCCTGTGACGCTTCAAAAGTACTGGCAGTTTACCGTCGACAGTGTGACGATCGG
TGGTGTTGTCGTTGCCTGCGACGGCGGTTGCCAAGCGATTCTGGATACCGGCACCTCAATGCTTGTGGGCCCTTCGAGTGATATTC
TGAACATCCAGATGGCGATAGGGGCCACACAGAACCAATATGGGGAGTTCGACATTGACTGTGGTTCGCTTAGTTCGATGCCCACC
GTGGTGTTCGAGATCAACTCACGCATGTACCCGCTCACACCTAGCGCATATACAAATCAGGACCAGGGTTTCTGTACAAGCGGTTT
TCAGGGAGAAAACAATTCCCAGCAATGGATCCTTGGGGACGTATTCATAAGGGAGTATTACTCTGTGTTTGATCGGGCCAACAACA
GAGTCGGTCTGGCTAAGGCCGTT
SEQ ID NO: 13
TCGGGCATCACACGAGTGCCACTTCTGAAAGGGAAGTCGTTGAGAAAGTCACTCAAAGAGCACGGACTCTTGGAGGAGTTCCTGCA
AACGCATGCCCGTAAAAAGCTCGCCCACTCTGGGAAGATCGCGCGAGAGCCGCTCACAAATTATTTGGACACGGAGTACTTCGGCA
AAATCCAGATCGGTAGCCCGCCTCAAGAGTTTACAGTCGTGTTCGACACCGGTTCTTCGGATCTCTGGGTTCCTTCCGTCTTTTGC
AATAGTGATGCCTGTCAGAACCATCATCGCTTTAATCCATCCAAGTCTAGCACCTTCCAAAACATGAACCGCCCCCTGAGTATCCA
ATACGGCACCGGTAGTATGCAGGGGTTCTTGGGTTATGATACAGTCACCGTATCGAACATCGTGGTTCCACACCAAACCGTAGGAT
TGAGCACTCAGGAACCTGGCGACATTTTCACTTACTCCGAATTCGATGGCATCCTCGGACTGGCGTACCCGTCCCTGGCTTCACAA
TATTCTGTGCCCGTCTTTGACAACATGATGAAGCAGCACCTCGTGGCTCAAGACCTTTTCTCTGTTTATATGAATAGCCAGGGACA
GGGTTCCATGCTGATACTGGGGGCTATTGACCCCTCATACTACGTCGGAGCACTTCATTGGGTCCCTGTGACGGTTCAGCAGTATT
GGCAGTTCACCGTCGACCGGATAACCGTGGATGGAGTTGTCGTGGCCTGCGACGGAGGTTGTCAAGCGATTCTTGATACCGGCACA
TCACTCCTCGTTGGGCCCTCCTCTGATATATTGAACATCCAGACGGTTATCGGCGCAACCCAGGGCCAATACGGGGAATTCGACAT
TGATTGCGGTTCCCTGAGATCGAAGCCAACGGTGGTATTCGAGATTAATGGTCGCGAATACCCGCTCCCGCCCACTGCTTATACTA
ATCAGGACCAGGGCTTCTGCACTTCGGGTTTTCAAGGAGATGATAGCCAGCAATGGGTTCTTGGCGACGTGTTTATCCGGGAATAC
TATTCCGTATTTGATCGTGTCAACAACAGGGTCGGCCTGGCAAAGGCCATT
SEQ ID NO: 14
TCCATCATCAGCCGAATCCCGCTTCACAAAGGAAAGAGTCTGCGGAAGGCGCTCAAAGAGCGCGGACTTCTGGAGGACTTTTTGAA
AAACCATCAGTACGCTGTCTCAAGGAAGCACTCATCGTCCGGGAAGGTCGCAAGAGAGAGCCTTACCAACTACCTGGATTGCCAGT
ACTTCGGGAAGATATACATTGGCACACCGCCCCAAGAGTTCACCGTGGTTTTCGACACTGGCAGTTCAGACCTCTGGGTGCCATCT
GTCTACTGCAATTCTGATGCCTGTCAGAATCATCATCGCTTCGACCCGTCTAAGTCGTCCACATTTCAAAACATGGATAAGTCGCT
GTCTATCCAATATGGTACCGGTTCGATGCAGGGTCTTTTGGGCTATGACACAGTTACTGTCTCAAATATTGTCGATCGCCACCAGA
CGGTCGGCCTGAGCACCCAAGAGCCCGGCGACGTGTTCACTTACTCTGAATTCGATGGCATCCTCGGACTGGCCTACCCCTCTCTC
GCGAGCGAATACTCGGTACCAGTGTTCGATAACATGATGGATAGGCACCTGGTCGCCCAAGATCTCTTCTCCGTCTATATGAGTCG
GAACGAGCAGGGTTCCATGTTGACTCTCGGGGCTATAGATCGATCCTATTACACGGGGTCGTTGCATTGGATCCCAGTGACGGTTC
AGGAGTACTGGCAGTTTACGGTCGATCGGGTTACCGTGGACGGCGTGGTCGTAGCCTGTGATGGCGGATGCCAGGCTATTTTGGAC
ACCGGTACCAGCATGCTCGTGGGACCGGGTTCAGACATTTTCAATATTCAGCAAGCCATCGGTGCAACCGAGGGACAATTTGGGGA
GTTCGACATCGATTGCGGCAGACTTAGCTCTATGCCCACTGTTGTCTTTGAAATCAACGGGAAAAAGTACCCTCTCCCTCCTTCCG
CTTATACGAGTCAGGACCAGGGCTTTTGTACATCGGGTTTTCAAGGAGATTATTCAAGCCAGCAATGGATTCTTGGCGACGTATTC
ATACGTGAATACTATTCCGTTTTCGACCGCGCAAGCAACCGTGTTGGATTGGCGAAAGCTATC
SEQ ID NO: 15
GCTTTTCGTCGGATCCCTCTGACCAAGGGAAAAACGCTGCGCAAGGTTCTTAAGGAGCATGGGCTTCTGGAGAGTTTTCTCAAGAG
CCACAAGTATTCTCCTTCTTCGAAATATCAGCTTTATGGGGAAGCAGCTAAGGTCACCGACGAACCCCTGACAAATTACCTCGACT
CGCAATACTTTGGCAAAATCTACATTGGGACACCACCGCAGGAATTCACTGTCGTATTTGATACCGGCTCATCCAACCTTTGGGTG
CCGTCTGTCTACTGCAATTCGGATGCGTGTCAGAACCATCACCGATTCAATCCAGCCTCGTCTACTACCTTCCGATCAACCCAGGA
GCCGCTCAGTATTCAGTATGGCACCGGCTCAATGGAAGGCGTCCTCGGTTACGATACAGTCACTGTCAGTCAAATTGTTGTGCCTG
ATCAGATCTTCGGACTCTCGACCCAAGAGCCTGGGGAGATCTTCACGTACTCCGAGTTCGACGGGATCCTGGGTCTGGGATACCCC
TCCTTGGCGGAGGATCAGGCCACTCCAGTTTTCGACAATATGATGAACAAAAACCTCGTGGCCCAAGATCTCTTCTCTGTCTATAT
GAGCAGGGACTCCCAGGGTTCGATGCTGATTCTTGGCGCGATAGATCCCTCCTATTACACGGGTTCACTGCATTGGGTGCCCGTTA
CGGAGCAGGGATATTGGCAGTTCAGTGTCGATTCAATTACCGTCAACGGCCAGGTTGTGGCTTGTGAAGGCGGTTGTCAAGCTATC
TTGGACACTGGCACAAGCCTCCTCGTTGGACCGAGCTACGATATCGCCAACATCCAATCCATTATCGGAGCCACCCAGGGCCAATA
CGGAGAATATGACATCAACTGCAGCAACCTGAGCAGTATGCCCACGGTGGTAGTCCACATAAACGGGCGTCAATACCCACTTCCTC
CCTCCGCGTATACTAATCAGGATCAGGGTTTGTGCTCCTCCGGTTTTCAATCTGAGGGCTCAGATCAGTTGTGGATCTTGGGGGAC
GTGTTTATAAGAGAGTACTACTCGGTGTTCGACCGGGGAAACAATCGCGTCGGTCTGGCAACGGCAGTA
SEQ ID NO: 16
ACTGGTATTACCCGCATTCCTCTGCACAAGGGCAAACCATTGCGCAAAGCCCTTAAGGAACACGGCTTGCTCGAGGATTTCCTGCA
AAAGCATCAGAGCGCAGTCTCGTCCAAGTATAGCTCTTTTGGGGAAGTAGCTTCAGAGCCGTTGACGTCATACCTGGACAGTCAGT
ACTTCGGAAAGATCTACATCGGAACGCCGCCCCAGGAATTCACCGTCGTTTTCGACACTGGCTCCTCGGATTTGTGGGTACCCTCT
GTTTACTGCAAAAGCGATGCATGCCAAAATCATCAGCGTTTTGATCCATCAATGTCGAGTACATTCCAGAATCTCGGGAAGCCCTT
GTCTATCCAATACGGCACCGGATCTATGCAGGGCTTCCTCGGTTATGATACCGTCACTGTGTCCGACATAGTCGATCCACAACAGA
CTGTCGGCCTCTCCACACAAGAGCCTGGGGATGTGTTCACTTACTCGGAATTTGACGGAATCTTGGGCCTGGCGTATCCGAGCCTC
GCTTCCGAATACTCCGTCCCCGTTTTCGATAACATGATGAATCGGCATCTGGTTGCTCAAGACCTCTTCTCCGTCTATATGGATCG
AAATGGACAAGAGTCGATGCTTACGCTGGGAGCTATCGACCCGAGCTACTATACTGGATCTCTCCACTGGGTCCCTGTGACGCTTC
AAAAGTACTGGCAGTTTACCGTCGACAGTGTGACGATCGGTGGTGTTGTCGTTGCCTGCGACGGCGGTTGCCAAGCGATTCTGGAT
ACCGGCACCTCAATGCTTGTGGGCCCTTCGAGTGATATTCTGAACATCCAGATGGCGATAGGGGCCACACAGAACCAATATGGGGA
GTTCGACATTGACTGTGGTTCGCTTAGTTCGATGCCCACCGTGGTGTTCGAGATCAACTCACGCATGTACCCGCTCACACCTAGCG
CATATACAAATCAGGACCAGGGTTTCTGTACAAGCGGTTTTCAGGGAGAAAACAATTCCCAGCAATGGATCCTTGGGGACGTATTC
ATAAGGGAGTATTACTCTGTGTTTGATCGGGCCAACAACAGAGTCGGTCTGGCTAAGGCCGTT
SEQ ID NO: 17
GEVASVPLTNYLDSQYFGKIYLGTPPQEFTVLEDTGSSDFWVPSIYCKSNACKNHQRFDPRKSSTFQNLGKPLSIHYGTGSMQGIL
GYDTVTVSNIVDIQQTVGLSTQEPGDVFTYAEFDGILGMAYPSLASEYSIPVFDNMMNRHLVAQDLFSVYMDRNGQESMLTLGAID
PSYYTGSLHWVPVTVQQYWQFTVDSVTISGVVVACEGGCQAILDTGTSKLVGPSSDILNIQQAIGATQNQYGEFDIDCDNLSYMPT
VVFEINGKMYPLTPSAYTSQDQGFCTSGFQSENHSQKWILGDVFIREYYSVFDRANNLVGLAKAI
SEQ ID NO: 18
GKVAREPLTSYLDSQYFGKIYIGTPPQEFTVVFDTGSSDLWVPSIYCKSNVCKNHHRFDPRKSSTFRNLGKPLSIHYGTGSMEGFL
GYDTVTVSNIVDPNQTVGLSTEQPGEVFTYSEFDGILGLAYPSLASEYSVPVFDNMMDRHLVARDLFSVYMDRNGQGSMLTLGAID
PSYYTGSLHWVPVTLQQYWQFTVDSVTINGVAVACVGGCQAILDTGTSVLFGPSSDILKIQMAIGATENRYGEFDVNCGNLRSMPT
VVFEINGRDYPLSPSAYTSKDQGFCTSGFQGDNNSELWILGDVFIREYYSVFDRANNRVGLAKAI

### REFERENCES

### Non-Patent Literature

L. K. Creamer, N. F. Olsen, J. Food Sci. 1982, 47:631-636
N. Bansal, M. A. Drake, P. Piraino, M. L. Broe, M. Harboe, P. F. Fox, P. L. H. McSweeney, Int. Dairy J. 2009, 19:510-517
A. C. Moynihan, S. Govindasamy-Lucey, J. J. Jaeggi, M. E. Johnson, J. A. Lucey, P. L. H. McSweeney, J. Dairy Sci. 2014, 97:85-96
P. L. McSweeney, Cheese manufacture and ripening and their influence on cheese flavor (2007), Improving the Flavour of Cheese, pages 1-25, from Woodhead Publishing Series in Food Science, Technology and Nutrition
Needleman S B, Wunsch C D, A general method applicable to the search for similarities in the amino acid sequence of two proteins, J Mol Biol. 1970 March; 48(3):443-53
EMBOSS: The European Molecular Biology Open Software Suite, Rice et al., 2000, Trends Genet. 16:276-277
Smith C A, Want E J, O'Maille G, Abagyan R, Siuzdak G. XCMS: processing mass spectrometry data for metabolite profiling using nonlinear peak alignment, matching, and identification. Anal Chem. 2006 Feb. 1; 78(3):779-87

### Patent Literature

WO2013174840, WO2013164479, WO2015128417, WO2016207214, WO2017037092, WO2017198810 or WO2017198829

1. A DNA sequence encoding a polypeptide, wherein the DNA sequence comprises:
a nucleotide sequence having at least 80% sequence identity with SEQ ID NO: 9 or SEQ ID NO: 13;
a nucleotide sequence having at least 80% sequence identity with SEQ ID NO: 10 or SEQ ID NO: 14;
a nucleotide sequence having at least 80% sequence identity with SEQ ID NO: 11 or SEQ ID NO: 15; or
a nucleotide sequence having at least 80% sequence identity with SEQ ID NO: 12 or SEQ ID NO: 16;
wherein the DNA sequence is an isolated DNA sequence or a recombinant DNA sequence or a synthetic DNA sequence; and/or
wherein the polypeptide has aspartic protease activity or chymosin activity or is an aspartic protease (EC 3.4.23) or is a chymosin (EC 3.4.23.4).

2. The DNA sequence according to claim 1, further encoding a signal peptide sequence and/or a linker sequence and/or a sequence encoding for a glucoamylase.

3. A vector comprising a DNA sequence according to any of the previous claims.

4. A host cell comprising a DNA sequence according to claim 1.

5. The host cell according to claim 4, wherein the host cell is selected from Aspergillus, Bacillus, or Pichia.

6. A polypeptide encoded by a DNA sequence according to claim 1, wherein the polypeptide has a higher specific clotting activity (C)/proteolytic activity (P) ratio than SEQ ID NO: 17 and/or SEQ ID NO: 18; and/or wherein polypeptide cleaves alpha-casein with a higher frequency than SEQ ID NO: 17 and/or SEQ ID NO: 18; and/or wherein the polypeptide cleaves alphaS1-casein with a higher frequency than SEQ ID NO: 17 and/or SEQ ID NO: 18, wherein alphaS1-casein cleavage is determined by quantifying alphaS1-casein peptides obtained in cheese or cheese sample prepared with the peptide or polypeptide or SEQ ID NO: 17 or SEQ ID NO: 18, wherein quantification is carried out by RP-HPLC coupled to an ESI-Q-TOF mass spectrometer.

7. A milk clotting composition comprising a polypeptide according to claim 6 or a polypeptide comprising:
an amino acid sequence having at least 70% sequence identity with SEQ ID NO: 1 or SEQ ID NO: 5;
an amino acid sequence having at least 70% sequence identity with SEQ ID NO: 2 or SEQ ID NO: 6;
an amino acid sequence having at least 70% sequence identity with SEQ ID NO: 3 or 7; or
an amino acid sequence having at least 70% sequence identity with SEQ ID NO: 4 or SEQ ID NO: 8.

8. A method for preparing a food product, wherein the method comprises:
adding a polypeptide comprising an amino acid sequence having at least 70% identity with SEQ ID NO: 1 or SEQ ID NO: 5, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 1 or SEQ ID NO: 5; or
adding a polypeptide comprising an amino acid sequence having at least 70% identity with SEQ ID NO: 2 or SEQ ID NO: 6, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 2 or SEQ ID NO: 6; or
adding a polypeptide comprising an amino acid sequence having at least 70% identity with SEQ ID NO: 3 or 7, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 3 or SEQ ID NO: 7; or
adding a polypeptide comprising an amino acid sequence having at least 70% identity with SEQ ID NO: 4 or SEQ ID NO: 8, preferably at least 75%, at least 78%, at least 80%, at least 83%, at least 85%, at least 90%, at least 91%, at least 92%, at least 93%, at least 94%, at least 95%, at least 96%, at least 97%, at least 98%, at least 99% or 100% sequence identity with SEQ ID NO: 4 or SEQ ID NO: 8 to milk; and
carrying out appropriate steps for preparing the food product.

9. The method according to claim 8, wherein the food product is a dairy product selected from a cheese, a cheese product, a processed cheese, a cheese-like product such as vegan cheese or non-dairy cheese, a butter, a yogurt, a cream, and a seasoning.

10. The method according to claim 8, wherein the food product or dairy product is a cheese or a cheese product, preferably wherein the cheese or cheese product is Cheddar cheese or Continental cheese or Gouda cheese.

11. The method according to claim 8, wherein the milk is selected from cow's milk, camel's milk, buffalo milk, goat's milk, sheep's milk, and/or a mixture thereof.

12. The method according to claim 8, wherein the polypeptide has a higher specific clotting activity (C)/proteolytic activity (P) ratio than a bovine chymosin, such as SEQ ID NO: 17; and has a higher degradation of alpha-casein or alphaS1-casein than a camel chymosin, such as SEQ ID NO: 18.

13. (canceled)

14. (canceled)

15. (canceled)
